(19) United States
(12) Patent Application Publication
(10) Pub. No.: US 2019/0307762 A1
(43) Pub. Date: Oct. 10, 2019

(54) METHODS AND COMPOSITIONS FOR THE TREATMENT OF TRAUMA AND STRESSOR-RELATED DISORDERS

(71) Applicant: Akhil MEHRA, San Francisco, CA (US)

(72) Inventor: Akhil MEHRA, San Francisco, CA (US)

(21) Appl. No.: 16/308,037

(22) PCT Filed: Jun. 2, 2017
(86) PCT No.: PCT/US2017/035609

§ 371 Date: Dec. 7, 2018

Related U.S. Application Data
(60) Provisional application No. 62/346,617, filed on Jun. 7, 2016.

(51) Int. Cl. A61K 31/55; A61K 31/573; A61P 25/22

(52) U.S. Cl. CPC A61K 31/55; A61P 25/22; A61K 31/573

(57) ABSTRACT

The present invention relates to methods for the treatment or prevention of symptoms associated with trauma and stressor-related disorders, in which exposure to a traumatic or stressful event is listed explicitly as a diagnostic criterion. These include reactive attachment disorder, disinhibited social engagement disorder, post-traumatic stress disorder (PTSD), acute stress disorder, and adjustment disorders. The present invention also relates to pharmaceutical compositions comprising opipramol alone, or in combination with another agent for the treatment or prevention of symptoms associated with trauma and stressor-related disorders. The present invention also relates to transdermal formulations of pharmaceutical compositions comprising opipramol alone, or in combination with another agent, for the treatment or prevention of symptoms associated with trauma and stressor-related disorders.

### BACKGROUND OF THE INVENTION

### 1. Field of the Invention

The present invention relates to methods for the treatment or prevention of symptoms associated with trauma and stressor-related disorders, in which exposure to a traumatic or stressful event is listed explicitly as a diagnostic criterion. These include reactive attachment disorder, disinhibited social engagement disorder, post-traumatic stress disorder (PTSD), acute stress disorder, and adjustment disorders. The present invention also relates to pharmaceutical compositions comprising opipramol alone, or in combination with another agent for the treatment or prevention of symptoms associated with trauma and stressor-related disorders. The present invention also relates to transdermal formulations of pharmaceutical compositions comprising opipramol alone, or in combination with another agent, for the treatment or prevention of symptoms associated with trauma and stressor-related disorders.

### 2. Description of the Related Art

Psychological distress following exposure to a traumatic or stressful event is quite variable. In some cases, symptoms can be well understood within an anxiety- or fear-based context. It is clear, however, that many individuals who have been exposed to a traumatic or stressful event exhibit a phenotype in which, rather than anxiety- or fear-based symptoms, the most prominent clinical characteristics are anhedonic and dysphoric symptoms, externalizing angry and aggressive symptoms, or dissociative symptoms. It is not uncommon for the clinical picture to include some combination of the above symptoms (with or without anxiety- or fear-based symptoms). Such a heterogeneous picture has long been recognized in adjustment disorders, as well. Social neglect that is, the absence of adequate caregiving during childhood may also generate similar types of symptoms. This indicates that a separate psychobiological process, pathologically distinct from that seen in conventional anxiety disorders, may be operating in the trauma and stress related disorders.

Acute stress disorder is a result of a traumatic event in which the person experienced or witnessed an event that involved threatened or actual serious injury or death and responded with intense fear and helplessness. Symptoms include dissociative symptoms such as numbing, detachment, a reduction in awareness of the surroundings, derealization, or depersonalization, re-experiencing of the trauma, avoidance of associated stimuli, and significant anxiety, including irritability, poor concentration, difficulty sleeping, and restlessness. If left untreated, the condition may evolve into Post Traumatic Stress Disorder (PTSD).

Post-traumatic stress disorder (PTSD) is a prevalent and highly debilitating psychiatric disorder that is notoriously difficult to treat. PTSD is characterized by flashbacks, emotional numbness, and insomnia, and is associated with functional impairments, physical health concerns, and mental health comorbidities, such as depression, with six fold higher risk of suicide. PTSD can result from a catastrophic and threatening event, e.g., a natural disaster, wartime situation, accident, domestic abuse, or violent crime. Symptoms typically develop within three months, but can emerge years after the initial trauma.

The treatment of PTSD is extremely challenging, and may include many years of individual and group therapy and medications such as antidepressants, anxiolytic drugs, beta-adrenergic antagonists, opiates, or cortisol with variable results. Selective serotonin reuptake inhibitors (SSRIs) are currently recommended as the first-line pharmacotherapy. However, up to 40% of S SRI-treated PTSD patients do not respond and >70% never achieve full remission.

Opipramol is a tricyclic compound with the nucleus of the anticonvulsant carbamazepine and the side chain of the neuroleptics fluphenazine and perphenazine without reuptake inhibiting properties for serotonin (5-HT) or noradrenaline. In vitro, it blocks the following receptors in decreasing order: histamine (H1.H2), serotonin (5-HT2), dopamine (D2.D1), adrenergic (al), and very weakly cholinergic receptors. The blocking potential for H1, 5-HT2 and D2 receptors places opipramol between the classical antidepressants, atypical neuroleptics and anxiolytics (classical sedatives as well as serotonergic antagonists in the developmental state). Recent basic research also characterized opipramol as a strong sigma ligand with complex interactions on the dopaminergic system and the NMDA receptor complex. It also induces increased levels of cGMP and it possesses anti-ischemic effects.

Opipramol was originally developed in the sixties as an antidepressant, its anxiolytic properties were soon recognized and have recently been confirmed in a well-designed clinical trial in patients with generalized anxiety disorders, demonstrating superiority over placebo and similar efficacy to alprazolam. Opipramol is clinically used in Germany and some other European countries with increasing tendency as a substitute for benzodiazepines, also because there is no risk of drug dependence.

There is therefore a need in the art for the discovery of additional methods for the treatment or prevention of symptoms associated with trauma and stressor-related disorders including PTSD. The present disclosure describes compositions and methods of using opipramol to treat PTSD. The methods of the present invention are directed to a distinct patient population characterized by a disease state different from that related to those disclosed in the prior art.

### SUMMARY OF THE INVENTION

Compositions and methods for the treatment or prevention of symptoms associated with trauma and stressor-related disorders, in which exposure to a traumatic or stressful event is listed explicitly as a diagnostic criterion, are provided.

The present invention relates to compositions and methods for treating disorders including reactive attachment disorder, disinhibited social engagement disorder, post-traumatic stress disorder (PTSD), acute stress disorder, and adjustment disorders.

The method of the present invention is intended for the alleviation of symptoms associated with trauma and stressor-related disorders, in which exposure to a traumatic or stressful event is listed explicitly as a diagnostic criterion. These include reactive attachment disorder, disinhibited social engagement disorder, posttraumatic stress disorder (PTSD), acute stress disorder, and adjustment disorders. The process involves administration of opipramol or a pharmaceutically acceptable acid addition salt thereof, to one in need of such treatment. Administration in single or divided doses may be anticipated as being the preferred dosage regimen. While various centrally-acting drug treatments have been employed for symptomatic relief in trauma and stress related disorder patients, it has now been found that opipramol possesses important clinical distinctions from previous treatments because of its differentiated mechanism of action. It is believed that this novel pharmacology results in unique therapeutic activity in treatment of trauma and stress related disorders.

Non anxiety/fear based symptoms in trauma and stressor related disorders may be caused by states of excess of the excitatory neurotransmitter glutamate. Clinical studies suggest that NMDA antagonists may transiently stimulate glutamate release and produce a syndrome similar to that seen in trauma and stressor related disorders. Drugs that block gluatmate release or protect cells against glutamate mediated excitatory neurotoxicity are thus candidates as treatments for trauma and stress related disorders.

In preclinical studies, linked to its ability to bind to sigma one receptors, opipramol has shown the ability to protect neurological tissue against the effects of glutamate mediated toxicity. In addition, opipramol may have unexpected benefits given this mechanism in trauma and stressor-related disorders. Opipramol also acts to down regulate signaling at sigma 2 receptors. Although the function of sigma 2 receptors is poorly characterized, similar sigma 2 active compounds have been shown to be powerful anxiolytic, as has opipramol in conventional anxiety disorders such as generalized anxiety disorder that do not involve aspects of dissociative phenomena. This combination of action on core symptoms sets of both fear and non fear/anxiety based symptoms (for example dissociative or dysphoric symptoms) make opipramol a uniquely and unexpectedly powerful treatment for trauma and stressor related disorders

One embodiment of the invention provides for methods for the treatment or prevention of symptoms associated with trauma and stressor-related disorders, in which exposure to a traumatic or stressful event is listed explicitly as a diagnostic criterion, by administering a therapeutically effective dosage of opipramol or a pharmaceutically acceptable salt or ester thereof.

In one aspect, the present disclosure provides a means for preventing, treating, ameliorating, alleviating and reducing signs and symptoms associated with trauma and stressor-related disorders, in which exposure to a traumatic or stressful event is listed explicitly as a diagnostic criterion, including acute stress disorder (ASD) and post traumatic stress disorder (PTSD) in mammalian subjects including humans.

These and other subjects are effectively treated by administering to the subject an effective amount of opipramol. The opipramol may be administered alone or with the addition of one or more additional psychotherapeutic agents in an amount effective to prevent, treat, ameliorate, alleviate or reduce disorders including reactive attachment disorder, disinhibited social engagement disorder, post-traumatic stress disorder (PTSD), acute stress disorder, and adjustment disorders.

In one aspect of the invention, means are provided herein for treating or preventing symptoms of a trauma and stressor-related disorder, in male and female subjects. These subjects are effectively treated by administering to the subjects an effective amount of opipramol or a pharmaceutically acceptable salt thereof. An exemplary opipramol as used herein is opipramol dihydrochloride (4-[3-(5H-dibenz[b,f]-azepine-5-yl)-propyl]-1-piperazine-ethanol dihydrochloride, CAS 315-72-0).

In another aspect of the invention, means are provided herein for treating or preventing symptoms of disorders including reactive attachment disorder, disinhibited social engagement disorder, post-traumatic stress disorder (PTSD), acute stress disorder, and adjustment disorders, in male and female subjects. These subjects are effectively treated by administering to the subjects an effective amount of opipramol or a pharmaceutically acceptable salt thereof. An exemplary opipramol as used herein is opipramol dihydrochloride.

The invention further provides means for preventing or treating signs and symptoms of disorders including reactive attachment disorder, disinhibited social engagement disorder, post-traumatic stress disorder (PTSD), acute stress disorder, and adjustment disorders, in mammalian subjects including humans, by administering an effective amount of a sustained release therapeutic agent. Such therapeutic agents include opipramol or a pharmaceutically acceptable salt thereof. The sustained release therapeutic agent typically will provide an increased bioavailability of the agent compared to an immediate release dosage form of the agent.

The invention further provides means of treating or preventing symptoms of the trauma and stressor-related disorder in a human subject who is either suffering from or at risk for trauma and stressor-related disorder by administering opipramol to the subject.

In another aspect of the invention, invention further provides means of treating or preventing symptoms of the trauma and stressor-related disorder in a human subject who is either suffering from or at risk for trauma and stressor-related disorder by controlled, transdermal administering opipramol to the subject.

In another aspect of the invention, treatment or prevention of symptoms of disorders including reactive attachment disorder, disinhibited social engagement disorder, post-traumatic stress disorder (PTSD), acute stress disorder, and adjustment disorders, is provided by administering an effective amount of a prodrug of opipramol to a human subject suffering from such symptoms. The prodrug of opipramol will advantageously provide for increased solubility and/or bioavailability compared to the parent drug of opipramol.

A further aspect of the invention is a opipramol prodrug. The opipramol prodrugs provided herein have increased solubility and/or bioavailability properties compared to the opipramol parent drug.

Within additional aspects of the invention, combinatorial formulations are provided which employ opipramol and psychotherapeutic agents effective to prevent, treat, ameliorate, alleviate or reduce the trauma and stressor-related disorder in the subject, including human subjects. Exemplary combinatorial formulations and coordinate treatment methods employ a psychotherapeutic agent including, but not limited to, drugs from the general classes of anti-depressant, mood-stabilizing, anxiolytic, anticonvulsant, antipsychotic, antiaddictive, appetite suppressant drugs, and opiate agonists. An exemplary opipramol as used herein is opipramol.

In the coordinate administration methods of the invention, the opipramol and the psychotherapeutic agent are administered concurrently or sequentially in any order to prevent or treat one or more symptoms of the targeted disorders including reactive attachment disorder, disinhibited social engagement disorder, post-traumatic stress disorder (PTSD), acute stress disorder, and adjustment disorders. When administered simultaneously, the opipramol and the psychotherapeutic agent may be combined in a single composition or combined dosage form, or administered at the same time in separate dosage forms.

The methods, formulations and coordinate treatment methods of the invention are effective to modulate, alleviate, treat or prevent one or more symptom(s) of the trauma and stressor-related disorder in a subject, including a mammalian subject. Such formulations and coordinate treatment methods may be administered prior to or shortly after a triggering event, or after development of symptoms of disorders including reactive attachment disorder, disinhibited social engagement disorder, post-traumatic stress disorder (PTSD), acute stress disorder, and adjustment disorders.

In order to determine whether an individual is at risk of acquiring ASD and/or PTSD and is therefore a candidate for preventative treatment with the present compositions and/or compounds, the individual's current life situation can be assessed. If the individual is at risk of exposure to a terrifying event or situation in which grave physical harm (including death, either to the individual or someone else) may occur or be likely to occur, or in which grave physical harm may be threatened, then the individual is a candidate for treatment with the present compounds and/or compositions in order to prevent ASD and/or PTSD. Traumatic events that may trigger ASD and PTSD include violent personal assaults, natural or human-caused disasters, accidents, and military combat.

If an individual has experienced such a traumatic event but has not yet exhibited symptoms of ASD or PTSD, the individual can also be treated with the present compounds and/or compositions. Preferably, an individual who has experienced a traumatic event but not yet exhibited symptoms of ASD or PTSD is treated within a week of exposure to such a traumatic event in order to effectively treat ASD and/or PTSD and prevent some or all of the symptomology associated with PTSD from occurring. More preferably, such an individual is treated within 24, 48, or 72 hours of exposure to the trauma, and even more preferably the individual is treated immediately following the event, i.e. within 1-6 hours of exposure to the traumatic event.

An individual who has already acquired ASD or PTSD can also be effectively treated with the present compounds and/or compositions. An individual who has acquired ASD or PTSD and who is therefore in need of treatment with the present compounds and/or compositions can be identified through the diagnosis of the individual by a skilled clinician, such as a psychologist or psychiatrist. Such a skilled clinician can make a diagnosis of PTSD by following the criteria contained in the DSM-IV, set forth in below:

Criteria for Post-Traumatic Stress Disorder

A. The person has been exposed to a traumatic event in which both of the following have been present:
(1) the person experienced, witnessed, or was confronted with an event or events that involved actual or threatened death or serious injury, or a threat to the physical integrity of self or others.
(2) the person's response involved intense fear, helplessness, or horror. Note: In children, this may be expressed instead by disorganized or agitated behavior.

B. The traumatic event is persistently reexperienced in one (or more) of the following ways:
(1) recurrent and intrusive distressing recollections of the event, including images, thoughts, or perceptions. Note: In young children, repetitive play may occur in which themes or aspects of the trauma are expressed.
(2) recurrent distressing dreams of the event. Note: In children, there may be frightening dreams without recognizable content.
(3) acting or feeling as if the traumatic event were recurring (includes a sense of reliving the experience, illusions, hallucinations, and dissociative flashback episodes, including those that occur upon awakening or when intoxicated). Note: In young children, trauma-specific reenactment may occur.
(4) intense psychological distress at exposure to internal or external cues that symbolize or resemble an aspect of the traumatic event.
(5) physiological reactivity on exposure to internal or external cues that symbolize or resemble an aspect of the traumatic event.

C. Persistent avoidance of stimuli associated with the trauma and numbing of general responsiveness (not present before the trauma), as indicated by three (or more) of the following:
(1) efforts to avoid thoughts, feelings, or conversations associated with the trauma.
(2) efforts to avoid activities, places, or people that arouse recollections of the trauma.
(3) inability to recall an important aspect of the trauma.
(4) markedly diminished interest or participation in significant activities.
(5) feeling of detachment or estrangement from others.
(6) restricted range of affect (e.g., unable to have loving feelings).
(7) sense of a foreshortened future (e.g., does not expect to have a career, marriage, children, or a normal life span).

D. Persistent symptoms of increased arousal (not present before the trauma), as indicated by two (or more) of the following:
(1) difficulty falling or staying asleep.
(2) irritability or outbursts of anger.
(3) difficulty concentrating.
(4) hypervigilance.
(5) exaggerated startle response.

E. Duration of the disturbance (symptoms in Criteria B, C, and D) is more than one month.

F. The disturbance causes clinically significant distress or impairment in social, occupational, or other important areas of functioning.

If an individual exhibits the appropriate combination of symptoms indicating a diagnosis of PTSD as outlined above, then that individual can be treated with the present compounds and/or compositions. In order to arrive at a diagnosis of PTSD, the patient's symptoms generally must significantly disrupt normal activities and last for more than one month. Diagnosis of another psychiatric disorder, such as depression, alcohol and drug abuse, or other anxiety disorder, may aid in diagnosis, as approximately 80 percent of patients with PTSD also have at least one other psychiatric disorder.

Both ASD and PTSD can be prevented or treated by administering therapeutically effective amounts of one or more of the present compounds and/or pharmaceutical compositions to a patient in need thereof. The present compounds and/or compositions are administered to a patient in a quantity sufficient to treat or prevent the symptoms and/or the underlying etiology associated with ASD or PTSD in the patient. The present compounds can also be administered in combination with other agents known to be useful in the treatment of PTSD, such as paroxetine and sertraline, either in physical combination or in combined therapy through the administration of the present compounds and agents in succession (in any order).

Administration of the present compounds and compositions can begin immediately following exposure to a traumatic event, preferably within the first week following the traumatic event, and more preferably within the first 24-72 hours. Administration of the compositions and compounds can alternatively begin prior to an anticipated traumatic event (such as impending combat), in order to prevent or reduce the severity of subsequent ASD and/or PTSD. The present compounds and compositions can also be administered following a subject's experience of symptoms of ASD and/or PTSD, such as during either the acute, chronic, or delayed-onset phase. The present invention thus includes the use of the present compounds and/or a pharmaceutical composition comprising such compounds to prevent and/or treat ASD or PTSD.

Depending upon the particular needs of the individual subject involved, the present compounds can be administered in various doses to provide effective treatments for PTSD. Factors such as the activity of the selected compound, half life of the compound, the physiological characteristics of the subject, the extent or nature of the subject's disease or condition, and the method of administration will determine what constitutes an effective amount of the selected compounds. Generally, initial doses will be modified to determine the optimum dosage for treatment of the particular subject. The compounds can be administered using a number of different routes including oral administration, topical administration, transdermal administration, intraperitoneal injection, or intravenous injection directly into the bloodstream. Effective amounts of the compounds can also be administered through injection into the cerebrospinal fluid or infusion directly into the brain, if desired.

In another embodiment, provided herein is a transdermal patch comprising the composition comprising opipramol and a pharmaceutically acceptable carrier suitable for transdermal or topical administration, wherein the carrier may comprise a skin penetration enhancer. Such a transdermal patch may be formulated to provide substantially continuous delivery of the opipramol to a patient.

These and other features are explained more fully in the embodiments illustrated below. It should be understood that in general the features of one embodiment also may be used in combination with features of another embodiment and that the embodiments are not intended to limit the scope of the invention.

### DETAILED DESCRIPTION

### Definitions

As used herein, an “active therapeutic agent” of the present invention includes opipramol and optionally includes one or more other psychotherapeutic compound.

The term “acute stress disorder” and “ASD” refers to a psychiatric condition in its broadest sense, as defined in American Psychiatric Association: Diagnostic and Statistical Manual of Mental Disorders, Fifth Edition, Washington, D.C., 2000 (“DSM-5”). The DSM-5 defines “acute stress disorder” as characterized by anxiety, dissociative, and other symptoms occurring within 1 month after exposure to an extreme traumatic stressor. The DSM-5 sets forth a generally accepted standard for diagnosing and categorizing acute stress disorder.

The term “administration” or “administering” includes routes of introducing the compounds, or a composition thereof, of the invention to a subject to perform their intended function. Examples of routes of administration that may be used include injection (subcutaneous, intravenous, parenterally, intraperitoneally, intrathecal), oral, inhalation, rectal and transdermal. The pharmaceutical compositions may be given by forms suitable for each administration route. For example, these compositions are administered in tablets or capsule form, by injection, inhalation, eye lotion, ointment, suppository, etc. administration by injection, infusion or inhalation; topical by lotion or ointment; and rectal by suppositories. Oral administration is preferred. The injection can be bolus or can be continuous infusion. Depending on the route of administration, a compound described herein can be coated with or disposed in a selected material to protect it from natural conditions which may detrimentally affect its ability to perform its intended function. A compound or composition described herein can be administered alone, or in conjunction with either another agent as described above or with a pharmaceutically-acceptable carrier, or both. A compound or composition described herein can be administered prior to the administration of the other agent, simultaneously with the agent, or after the administration of the agent. Furthermore, a compound described herein can also be administered in a prodrug form which is converted into its active metabolite, or more active metabolite in vivo.

The terms “carrier” or “vehicle” as used herein refer to carrier materials suitable for transdermal drug administration. Contemplated carriers and/or vehicles include any such materials known in the art, which are substantially nontoxic and/or do not interact with other components of a pharmaceutical formulation or drug delivery system in a deleterious manner. Examples of specific suitable carriers and vehicles for use herein include water, propylene glycol, mineral oil, silicone, inorganic gels, aqueous emulsions, liquid sugars, waxes, petroleum jelly, and/or other oils and polymeric materials.

The term “continuously” or “continuous delivery” as used herein refers to a drug delivered substantially slowly and substantially uninterrupted for e.g. 2, 3, 8, 12, or more hours or even 1, 2, 3, 5, 7, 10, 12, 15, 30 or more days. In some embodiments, the term continuously refers to delivery of a drug or agent that is substantially longer as compared to bolus single or multiple doses. For this purpose, the transdermal patches according to the present disclosure are suitable.

The term “dissociation” or “dissociative disorder” as used herein refers to to the disconnection or lack of connection between things usually associated with each other. Dissociated experiences are not integrated into the usual sense of self, resulting in discontinuities in conscious awareness. In severe forms of dissociation, disconnection occurs in the usually integrated functions of consciousness, memory, identity, or perception. Dissociation may affect a person subjectively in the form of “made” thoughts, feelings, and actions. These are thoughts or emotions seemingly coming out of nowhere, or finding oneself carrying out an action as if it were controlled by a force other than oneself. There are five main ways in which the dissociation of psychological processes changes the way a person experiences living: depersonalization, derealization, amnesia, identity confusion, and identity alteration. The DSM-5 defines dissociation as “a disruption and/or discontinuity in the normal integration of consciousness, memory, identity, emotion, perception, body representation, motor control and behavior” (American Psychiatric Association, 2013, p. 291). The dissociative disorders include: dissociative amnesia (extensive forgetting typically associated with highly aversive events); dissociative fugue (short-lived reversible amnesia for personal identity, involving unplanned travel or “bewildered wandering.” Dissociative fugue is not viewed as a separate disorder but is a feature of some, but not all, cases of dissociative amnesia);

depersonalization/derealization disorder (feeling as though one is an outside observer of one's body); and dissociative identity disorder (DID; experiencing two or more distinct identities that recurrently take control over one's behavior) (American Psychiatric Association, 2000). The Structural Clinical Interview for DSM-IV Dissociative Disorders (SCID-D) (Steinberg, 2001) assesses five symptom clusters that encompass key features of the dissociative disorders. These clusters are also found in the DSM-5: depersonalization, derealization, dissociative amnesia, identity confusion, and identity alteration. The Dissociative Experiences Scale (DES) (Bernstein & Putnam, 1986; Carlson & Putnam, 2000; Wright & Loftus, 1999) is the most widely used self-report measure of dissociation.

The terms “glucocorticoid” and “glucocorticosteroid” as used herein refers to a therapeutically, prophylactically and/or diagnostically active glucocorticoid or a glucocorticoid that has physiologic effect. The term is intended to include the glucocorticoid in any suitable form such as e.g. a pharmaceutically acceptable salt, complex, solvate, ester, active metabolites or prodrug thereof of in any physical form such as, e.g., in the form of crystals, amorphous or a polymorphous form or, if relevant, in any stereoisomer form including any enantiomeric or racemic form, or a combination of any of the above. The glucocorticoid may be a synthetic glucocorticoid.

The terms, “individual,” “patient,” or “subject” are used interchangeably herein and include any mammal, including animals, for example, primates, for example, humans, and other animals, for example, dogs, cats, swine, cattle, sheep, rodents, and horses. The compositions disclosed herein can be administered to a mammal, such as a human, but can also be other mammals, for example, an animal in need of veterinary treatment, for example, domestic animals (for example, dogs, cats, and the like), zoo and wild animals, farm animals (for example, cows, sheep, pigs, horses, and the like) and laboratory animals (for example, rats, mice, guinea pigs, and the like).

As used herein, “opipramol” includes opipramol or a metabolite thereof, prodrugs of opipramol or a metabolite thereof. Metabolites of opipramol useful according to the methods of this invention are metabolites that have substantially the same activity or better as opipramol in alleviating symptom. A prodrug of opipramol is a derivative of opipramol that is metabolized in vivo into the active agent. Prodrugs useful according to this invention are those that have substantially the same activity or better than opipramol in treating or preventing the symptoms of trauma and stress related disorders. Methods for making prodrugs are readily known in the art (e.g., Balant, L. P., Prodrugs for the Improvement of Drug Absorption Via Different Routes of Administration, Eur. J. Drug Metab. Pharmacokinet. 15:143-153 (1990); and Bundgaard, H., Novel Chemical Approaches in Prodrug Design, Drugs of the Future 16:443-458 (1991); incorporated by reference herein).

The terms “penetration enhancement” or “permeation enhancement” as used herein refer to an increase in the permeability of skin to a pharmacologically active agent, i.e., so as to increase the rate at which the active agent permeates through the skin and enters the bloodstream. The enhanced permeation effected through the use of skin permeation enhancers, for example, through the use of a composition disclosed herein, can be observed by e.g., measuring the rate of diffusion of drug ex vivo, i.e., through animal or human skin using a diffusion cell apparatus, or in vivo, as described in the examples herein.

The terms “pharmaceutically acceptable” or “pharmacologically acceptable” refer to molecular entities and compositions that do not produce an adverse, allergic or other untoward reaction when administered to an animal, or to a human, as appropriate. The term “pharmaceutically acceptable carrier” includes any and all solvents, dispersion media, coatings, antibacterial and antifungal agents, isotonic and absorption delaying agents and the like. The use of such media and agents with pharmaceutical active agents is well known in the art. In some embodiments, supplementary active ingredients can also be incorporated into the compositions.

As used herein, the terms “prevention” and “preventing,” when referring to a trauma and stressor-related disorder or symptom, refers to a reduction in the risk or likelihood that a mammalian subject will develop the disorder, symptom, condition, or indicator after treatment according to the invention, or a reduction in the risk or likelihood that a mammalian subject will exhibit a recurrence of the disorder, symptom, condition, or indicator once a subject has been treated according to the invention and cured or restored to a normal state (e.g., placed in remission from a targeted disorder). As used herein, the terms “treatment” or “treating,” when referring to disorders, particularly acute stress disorder (ASD) and post traumatic stress disorder (PTSD), refers to inhibiting or reducing the progression, nature, or severity of the subject condition or delaying the onset of the condition.

The term “prodrug”, as used herein typically refers to a derivative of an active compound or drug that requires a transformation under the conditions of use, such as within the body, to release the active drug. Prodrugs are frequently, but not necessarily, pharmacologically inactive until converted into the active drug. Prodrugs are typically obtained by masking a functional group in the drug believed to be in part required for activity with a progroup to form a promoiety which undergoes a transformation, such as cleavage, under the specified conditions of use to release the functional group, and hence the active drug. The cleavage of the promoiety can proceed spontaneously, such as by way of a hydrolysis reaction, or it can be catalyzed or induced by another agent, such as by an enzyme, by light, by acid, or by a change of or exposure to a physical or environmental parameter, such as a change of temperature.

A wide variety of progroups, as well as the resultant promoieties, suitable for masking functional groups in active drugs, e.g., opipramol, to yield prodrugs, are useful targets. For example, a hydroxyl functional group can be masked as a sulfonate, ester or carbonate promoiety, which can be hydrolyzed in vivo to revert the hydroxyl group. An amino functional group can be masked as an amide, carbamate, imine, urea, phosphenyl, phosphoryl or sulfenyl promoiety, which can be hydrolyzed in vivo to revert the amino group. A carboxyl group can be masked as an ester (e.g., silyl esters and thioesters), amide or hydrazide promoiety, which can be hydrolyzed in vivo to revert the carboxyl group. Other examples of suitable progroups and their respective promoieties will be apparent to those of skill in the art.

The term “stress disorder” refers to a psychiatric condition precipitated by exposure to a traumatic or stressful event. stress disorders include acute stress disorder, post-traumatic stress disorder, and brief psychotic disorder with marked stressor(s). The term “brief psychotic disorder with marked stressor(s)” refers to a psychiatric condition in its broadest sense, as defined in DSM-IV-TR. The DSM-IV-TR defines “brief psychotic disorder with marked stressor(s)” as a sudden but brief onset of psychotic symptoms developing shortly after and apparently in response to one or more stressful events. The DSM-IV-TR sets forth a generally accepted standard for diagnosing and categorizing brief psychotic disorder with marked stressor(s).

As used herein, the term “sustained release vehicle, matrix, binder, or coating material” refers to any vehicle, matrix, binder, or coating material that effectively, significantly delays dissolution of the active compound in vitro, and/or delays, modifies, or extends delivery of the active compound into the blood stream (or other in vivo target site of activity) of a subject following administration (e.g., oral administration), in comparison to dissolution and/or delivery provided by an “immediate release” formulation, as described herein, of the same dosage amount of the active compound. Accordingly, the term “sustained release vehicle, matrix, binder, or coating material” as used herein is intended to include all such vehicles, matrices, binders and coating materials known in the art as “sustained release”, “delayed release”, “slow release”, “extended release”, “controlled release”, “modified release”, and “pulsatile release” vehicles, matrices, binders and coatings. As used herein, “sustained release” and “sustained delivery” are evinced by a sustained, delayed, extended, or modified, in vitro or in vivo dissolution rate, in vivo release and/or delivery rate, and/or in vivo pharmacokinetic value(s) or profile.

As used herein, a “therapeutically effective amount” of opipramol for the purposes of this invention refers to the amount of the compound that prevents or alleviates or eliminates or interferes with one of the symptoms associated with a trauma or stress related disorder. A physician can readily determine when symptoms are prevented or alleviated or eliminated, for example through clinical observation of a subject, or through reporting of symptoms by the subject during the course of treatment. One skilled in the art can readily determine an effective amount of opipramol to be administered, by taking into account factors such as the size, weight, age and sex of the subject, the extent of disease penetration or persistence and severity of symptoms, and the route of administration. In the case of therapeutic agents, these terms most often refer to a measurable, statistically significant reduction in an occurrence, frequency, or severity of one or more symptom(s) of a specified trauma and stressor-related disorder, including any combination of neurological and/or psychological symptoms, diseases, or conditions, associated with or caused by the targeted disorder.

The term “transdermal” refers generally to passage of an agent across the skin layers. For example, the term “transdermal” may refer to delivery of an agent (e.g., a vaccine or a drug) through the skin to the local tissue or systemic circulatory system without substantial cutting or penetration of the skin, such as cutting with a surgical knife or piercing the skin with a hypodermic needle. The term “transdermal delivery” refers to drug delivery across the skin, usually accomplished without breaking the skin. Transdermal delivery includes delivery via passive diffusion.

The term “treating” is used herein to denote treating the disease, disorder or condition, or ameliorating, alleviating, reducing, or suppressing symptoms of the disease, or slowing or stopping the progress of the disease. Thus, in some embodiments, administration of the composition or combination of the present disclosure may ameliorate, alleviate or reduce the cognitive disorder

### DESCRIPTION

In one aspect the invention is a method for treating or preventing any of the group of trauma and stress related disorders in which exposure to a traumatic or stressful event is listed explicitly as a diagnostic criterion. These include reactive attachment disorder, disinhibited social engagement disorder, posttraumatic stress disorder (PTSD), acute stress disorder, and adjustment disorders

The method comprises administering to a human in need of such treatment a pharmaceutical composition comprising of opipramol in a therapeutically effective amount and a therapeutically effective carrier. Opipramol, either alone or in combination with additional therapeutics, may be helpful in the expression of any of the listed trauma and stress related disorders

The symptoms may be a dissociative or non-dissociative symptom.

Stress disorders treatable by the methods of the present invention include, but are not limited to, acute stress disorder (ASD), post-traumatic stress disorder and brief psychotic disorder with marked stressor(s).

Acute Stress Disorder (ASD) is characterized by a constellation of symptoms, lasting at least two days, that appear and resolve within one month of exposure to an extreme traumatic stressor. If symptoms appear or persist beyond one month after exposure to the traumatic stressor, the patient may be considered to suffer from Post-Traumatic Stress Disorder rather than ASD. ASD is a common precursor to Post-Traumatic Stress Disorder, and up to 80% of trauma survivors initially suffering from ASD will meet the diagnostic criteria for Post-Traumatic Stress Disorder six months later (see Brewin et al., Am J Psychiatry 156:360-6, 1999).

Patients develop ASD following exposure to an extreme traumatic stressor (DSM-IV-TR Criterion A). A person must respond to the stressor with intense fear, helplessness, or horror to be diagnosed with ASD. ASD may develop from direct experience of traumatic events, including violent crimes, physical trauma, combat, diagnosis with a life-threatening illness, and natural or manmade disasters. Patients may also develop ASD from witnessing or learning about traumatic events that happen to others, especially family members or close friends. Unexpected exposure to death, dead bodies, or body parts may also induce ASD.

A diagnosis of ASD requires that the person meet several other symptomatic criteria. The person must experience at least 5, 6, 7, 8, 9 or more of the following symptoms from any of the five categories of intrusion, negative mood, dissociation, avoidance, and arousal, beginning or worsening after the traumatic event(s) occurred:
1. Intrusion Symptoms
   a) Recurrent, involuntary, and intrusive distressing memories of the traumatic event(s). Note: In children, repetitive play may occur in which themes or aspects of the traumatic event(s) are expressed.
   b) Recurrent distressing dreams in which the content and/or affect of the dream are related to the event(s). Note: In children, there may be frightening dreams without recognizable content.)
   Dissociative reactions (e.g., flashbacks) in which the individual feels or acts as if the traumatic event(s) were recurring. (Such reactions may occur on a continuum, with the most extreme expression being a complete loss of awareness of present surroundings.) Note: In children, traumaspecific reenactment may occur in play.
   d) Intense or prolonged psychological distress or marked physiological reactions in response to internal or external cues that symbolize or resemble an aspect of the traumatic event(s).
2. Negative Mood
   e) Persistent inability to experience positive emotions (e.g., inability to experience happiness, satisfaction, or loving feelings).
3. Dissociative Symptoms
   f) An altered sense of the reality of one's surroundings or oneself (e.g., seeing oneself from another's perspective, being in a daze, time slowing).
   g) Inability to remember an important aspect of the traumatic event(s) (typically due to dissociative amnesia and not to other factors such as head injury, alcohol, or drugs).
4. Avoidance Symptoms
   h) Efforts to avoid distressing memories, thoughts, or feelings about or closely associated with the traumatic event(s).
   i) Efforts to avoid external reminders (people, places, conversations, activities, objects, situations) that arouse distressing memories, thoughts, or feelings about or closely associated with the traumatic event(s).
5. Arousal Symptoms
   j) Sleep disturbance (e.g., difficulty falling or staying asleep, restless sleep).
   k) Irritable behavior and angry outbursts (with little or no provocation), typically expressed as verbal or physical aggression toward people or objects.
   l) Hypervigilance.
   m) Problems with concentration.
   n) Exaggerated startle response.

Finally, the diagnosis requires:
1. Duration of the disturbance (symptoms in Criterion B) is 3 days to 1 month after trauma exposure. Note: Symptoms typically begin immediately after the trauma, but persistence for at least 3 days and up to a month is needed to meet disorder criteria.
2. The disturbance causes clinically significant distress or impairment in social, occupational, or other important areas of functioning.
3. The disturbance is not attributable to the physiological effects of a substance (e.g., medication or alcohol) or another medical condition (e.g., mild traumatic brain injury) and is not better explained by brief psychotic disorder.

Like Acute Stress Disorder, Post-Traumatic Stress Disorder (PTSD) emerges following exposure to an extreme traumatic stressor, and is characterized by persistent reexperiencing of the traumatic event, avoidance of stimuli associated with the trauma, and anxiety or increased arousal. The types of traumatic stressors giving rise to PTSD, and the manifestations of PTSD symptoms, are identical to those described above for ASD, but for three differences. First, the dissociative symptoms required for a diagnosis of ASD are not required for a diagnosis of PTSD, although dissociative symptoms may commonly be seen in PTSD patients. Secondly, PTSD need not arise within one month of exposure to the traumatic stressor, and may emerge months or years after the traumatic event. Thirdly, in contrast to the one month maximum duration of symptoms required for a diagnosis of ASD, symptoms must persist for at least one month in order for a diagnosis of PTSD to be made.

A Brief Psychotic Disorder is a short-term (between one day and one month) disturbance involving the sudden onset of at least one psychotic symptom, such as delusions, hallucinations, disorganized speech, or grossly disorganized or catatonic behavior. Brief Psychotic Disorders exclude those induced by a general medical condition. If psychotic symptoms develop shortly after, and apparently in response to, one or more severely stressful events, the disturbance is diagnosed as Brief Psychotic Disorder with Marked Stressor(s) (formerly labeled “brief reactive psychosis” in DSM-III-R). Brief Psychotic Disorder with Marked Stressor(s) is treatable by the glucocorticoid receptor antagonists of the present invention.

The pharmaceutical composition may be in unit dosage form, e.g. as tablets or capsules. In such form, the composition is sub-divided in unit doses containing appropriate quantities of the active ingredient; the unit dosage forms can be packaged compositions, for example, packeted powders or vials or ampoules. The unit dosage form can be a capsule, cachet or tablet itself, or it can be the appropriate number of any of these in package form. The quantity of the active ingredient in a unit dose of composition may be varied or adjusted from 5 to 500 mg per day, from 25 to 450 mg per day, from 25 to 400 mg per day, from 50 to 400 mg per day, from 50 to 300 mg per day, from 100 to 300 mg per day, from 200 to 300 mg per day, according to the particular need and the activity of the active ingredient. The usual oral recommended dose of opipramol for humans may be from 5 to 500 mg per day, from 25 to 450 mg per day, from 25 to 400 mg per day, from 50 to 400 mg per day, from 50 to 300 mg per day, from 100 to 300 mg per day, from 200 to 300 mg per day and this dose may be administered in two or three divided doses. A maximum recommended daily dose for humans would be about 500 mg, 450 mg, 400 mg, 350 mg, 300 mg, or less but it will be understood by one skilled in the art that dosage under this invention will be determined by the particular circumstances surrounding each case. Higher or lower doses are also contemplated.

In one embodiment opipramol therapy can be carried out indefinitely to alleviate the symptoms of interest and frequency of dosage may be changed to be taken as needed. The period of treatment should be carried out for as long as necessary to alleviate one or more of the core symptoms of each of the trauma and stress related disorders mentioned.

In another embodiment of the invention, opipramol is administered in combination with a drug which may further alleviate the symptoms of the trauma or stress related disorder. The drugs may be administered sequentially or concurrently with the opipramol.

In another aspect, the invention may be employed for treating or preventing the development (either the initiation, consolidation or perpetuation) of a trauma or stress related disorder following a traumatic event. A traumatic event is defined as a direct personal experience that involves actual or threatened death or serious injury, or other threat to one's physical integrity; or witnessing an event that involves death, injury, or a threat to the physical integrity of another person; or learning about unexpected or violent death, serious harm, or threat of death or injury experienced by a family member or other close associate. Traumatic events that are experienced directly include, but are not limited to, military combat, violent personal assault (sexual assault, physical attack, robbery, mugging), being kidnapped, being taken hostage, terrorist attack, torture, incarceration as a prisoner of war or in a concentration camp, natural or manmade disasters, severe automobile accidents, or being diagnosed with a life-threatening illness. For children, sexually traumatic events may include developmentally inappropriate sexual experiences without threatened or actual violence or injury. Witnessed events include, but are not limited to, observing the serious injury or unnatural death of another person due to violent assault, accident, war, or disaster or unexpectedly witnessing a dead body or body parts. Events experienced by others that are learned about include, but are not limited to, violent personal assault, serious accident, or serious injury experienced by a family member or a close friend; learning about the sudden, unexpected death of a family member or a close friend; or learning that one's child has a life-threatening disease. The disorder may be especially severe or long lasting when the stressor is of human design (e.g., torture, rape).

The present invention provides novel methods and combined drug compositions, dosage forms, packages, and kits for the treatment or prevention of symptoms associated with trauma and stressor-related disorders, in which exposure to a traumatic or stressful event is listed explicitly as a diagnostic criterion.

The methods and compositions of the invention use of opipramol alone or in combination with other psychotherapeutic drugs to modulate, prevent, alleviate, ameliorate, reduce or treat the symptoms of disorders including reactive attachment disorder, disinhibited social engagement disorder, post-traumatic stress disorder (PTSD), acute stress disorder (ASD), and adjustment disorders. In some embodiments, administration of the compositions and methods of the present invention may prevent a trauma or stressor-related disorder including ASD and PTSD from developing. In other embodiments, administration of the compositions and methods of the present invention may prevent recurrent episodes of a trauma or stressor-related disorder

Subjects amenable to treatment according to the invention include mammalian subjects, including humans, suffering from or at risk for any of a variety of disorders including reactive attachment disorder, disinhibited social engagement disorder, post-traumatic stress disorder (PTSD), acute stress disorder, and adjustment disorders. Within the methods of the invention, opipramol is administered in an amount effective to treat a specified disorder alone or in combination with another psychotherapeutic drug including, but not limited to, drugs from the general classes of anti-depressant, mood-stabilizing, anxiolytic, anticonvulsant, antipsychotic, antiaddictive, appetite suppressant drugs and opiate agonists. (See, e.g., R J. Baldessarini in Goodman & Gilman's The Pharmacological Basis of Therapeutics, 9th Edition, Chapter 18, McGraw-Hill, 1996 for a review).

Exemplary formulations use an opipramol such as opipramol dihydrochloride alone or in combination with one or more psychotherapeutic drugs including, but not limited to, anti-depressants, mood-stabilizing, anxiolytic, anticonvulsant, antipsychotic, antiaddictive, appetite suppressant drugs and opiate agonists.

Dosing and therapeutic benefits of the opipramol coordinately administered with the psychotherapeutic drug will typically have similarly favorable therapeutic effects and comparable side effects as a therapeutic benefit and side effect profile achieved in control patients treated with the psychotherapeutic agent alone. However, in certain embodiments the dosage of the psychotherapeutic agent may be lowered and yet in combination with the opipramol, will still have comparable therapeutic benefits and similar side effects as a therapeutic benefit and side effect profile achieved in control patients treated with a higher dosage of the psychotherapeutic agent alone. Additionally, the opipramol, may also be present in lower or sub-therapeutic amounts yet in combination with the psychotherapeutic drug will still have comparable therapeutic benefits and similar side effects as a therapeutic benefit and side effect profile achieved in control patients treated with a higher dosage of the opipramol alone.

Within more detailed embodiments of the invention, the compositions and methods of the invention achieve substantial therapeutic benefit in terms of a clinical reduction in incidence, development, rate, recurrence, or severity of disorders including reactive attachment disorder, disinhibited social engagement disorder, post-traumatic stress disorder (PTSD), acute stress disorder, and adjustment disorders. In related embodiments, the compositions and methods of the invention measurably alleviate or prevent one or more symptoms of the trauma and stressor-related disorder.

In more detailed embodiments of the invention, anxiety disorders, which disorder is typically defined as an extended period (e.g. at least six months, except in the instance of acute stress disorder) of excessive anxiety or worry with symptoms on most days of this period, is treated. In embodiments of the invention, disorders including reactive attachment disorder, disinhibited social engagement disorder, post-traumatic stress disorder (PTSD), acute stress disorder, and adjustment disorders may be treated.

In one embodiment, the methods and compositions of the invention are employed to treat acute stress disorder which is a trauma and stress related disorder that can develop immediately following a traumatic event and lasts for no more than four weeks. Symptoms include re-experiencing the event, hyperarousal, avoidance, and dissociative symptoms such as feeling numb or attached. In some circumstances, it can evolve into PTSD.

The methods and compositions of the invention may also be used to treat PTSD. PTSD is characterized by the development of symptoms following exposure to an extreme traumatic stressor. The traumatic stressor must involve direct personal experience of an event that involves actual or threatened death or serious injury, or other threat to one's physical integrity; witnessing an event that involves death, injury, or a threat to the physical integrity of another person; or learning about unexpected or violent death, serious harm, or threat of death or injury experienced by a family member or other close associate.

Exemplary traumatic events include, but are not limited to military combat, violent personal assault, being kidnapped, being taken hostage, terrorist attack, torture, incarceration as a prisoner of war or in a concentration camp, natural or manmade disasters, severe automobile accidents, or being diagnosed with a life-threatening illness; observing the serious injury or unnatural death of another person due to violent assault, accident, war, or disaster or unexpectedly witnessing a dead body or body parts; learning about violent personal assault, serious accident, or serious injury experienced by a family member or a close friend; learning about the sudden, unexpected death of a family member or a close friend; or learning that one's child has a life-threatening disease.

Symptoms include, but are not limited to, persistent re-experiencing of the traumatic event, persistent avoidance of stimuli associated with the trauma and numbing of general responsiveness, and persistent symptoms of hyperarousal for more than 1 month. Additionally, the symptoms must cause clinically significant distress or impairment in social, occupational, or other important areas of functioning. The traumatic event can be re-experienced in various ways. Commonly the person has recurrent and intrusive recollections of the event or recurrent distressing dreams during which the event is replayed. In rare instances, the person experiences dissociative states that last from a few seconds to several hours, or even days, during which components of the event are relived and the person behaves as though experiencing the event at that moment. Intense psychological distress or physiological reactivity often occurs when the person is exposed to triggering events that resemble or symbolize an aspect of the traumatic event. Avoidance/numbing may include efforts to avoid thoughts, feelings, conversations about the traumatic event, activities, situations, or people who arouse recollections of it. In some instances, avoidances may include amnesia regarding the event. Numbing may manifest as markedly diminished interest or participation in previously enjoyed activities, feeling detached or estranged from other people, or of having markedly reduced ability to feel emotions. Hyperarousal may manifest as difficulty falling or staying asleep that may be due to recurrent nightmares during which the traumatic event is relived, hypervigilance, and exaggerated startle response. Some individuals report irritability or outbursts of anger or difficulty concentrating or completing tasks. (See 309.81 DSM-IV Criteria for Post-traumatic Stress Disorder).

It has been theorized that there may be multiple subtypes of patients with PTSD, including those with sensitized noradrenergic systems, sensitized serotonergic systems (Southwick et al., 1997), and endorphin system involvement (Holbrook et al., 2010). Investigations into PTSD have found alterations in the hypothalamic-pituitary-adrenocortical axis and in catecholaminergic and serotonergic systems (Marshall et al., 2001). In certain embodiments, the psychological response triggers one or more physical responses including, but not limited to, abnormal respiration, abnormal cardiac rhythm, abnormal blood pressure, or abnormal sensory processing. In particular, the methods and compositions of the invention may be used to treat PTSD in women, or men.

In the cases of anxiety disorder with external stimuli such as acute stress disorder or PTSD, the compositions and methods of the present invention may be used prophylactically. For example, the compositions of the present invention may be administered prior to exposure to a traumatic event or shortly after a traumatic event as well as after development of symptoms.

Transdermal administration of opipramol or the coordinate treatment method or combinatorial drug composition of the invention to suitable subjects (e.g., qualified subjects suffering from a trauma and stressor-related disorder or at increased risk for developing a trauma and stressor-related disorder) will yield a reduction in one or more target symptom(s) associated with the selected anxiety disorder or development of the anxiety disorder by at least 10%, 20%, 30%, 50% or greater, up to a 75-90%, or 95% or greater, compared to placebo-treated or other suitable control subjects. Comparable levels of efficacy are contemplated for the entire range of trauma and stressor-related disorder described herein, including all contemplated neurological and psychiatric disorders, and related conditions and symptoms, for treatment or prevention using the compositions and methods of the invention. These values for efficacy may be determined by comparing accepted therapeutic indices or clinical values for particular test and control individuals over a course of treatment/study, or more typically by comparing accepted therapeutic indices or clinical values between test and control groups of individuals using standard human clinical trial design and implementation.

Therapeutic efficacy can alternatively be demonstrated by a decrease in the frequency or severity of symptoms associated with the treated condition or disorder, or by altering the nature, occurrence, recurrence, or duration of symptoms associated with the treated condition or disorder. Therapeutic efficacy with the treated condition or disorder, or by altering the nature, recurrence, or duration of symptoms associated with the treated condition or disorder In this context, “effective amounts,” “therapeutic amounts,” “therapeutically effective amounts,” and “effective doses” of psychotherapeutic drugs and opipramols within the invention can be readily determined by ordinarily skilled artisans following the teachings of this disclosure and employing tools and methods generally known in the art, often based on routine clinical or patient-specific factors. In some embodiments, therapeutic efficacy will be determined prophylactically in that fewer or no subjects will develop the trauma and stressor-related disorder in comparison to the related population. For example, in the case of combat soldiers, fewer than 25% of the treated population would develop symptoms of PTSD.

Efficacy of the coordinate treatment methods and drug compositions of the invention will often be determined by use of conventional patient surveys or clinical scales to measure clinical indices of trauma and stressor-related disorder including ASD and PTSD in subjects. The methods and compositions of the invention will yield a reduction in one or more scores or selected values generated from such surveys or scales completed by test subjects (indicating for example an incidence or severity of a selected trauma and stressor-related disorder), by at least 10%, 20%, 30%, 50% or greater, up to a 75-90%, or 95% compared to correlative scores or values observed for control subjects treated with placebo or other suitable control treatment. In at risk populations, the methods and compositions of the invention will yield a stable or minimally variable change in one or more scores or selected values generated from such surveys or scales completed by test subjects. More detailed data regarding efficacy of the methods and compositions of the invention can be determined using alternative clinical trial designs.

Useful patient surveys and clinical scales for comparative measurement of clinical indices of trauma and stressor-related disorder in subjects treated using the methods and compositions of the invention can include any of a variety of widely used and well known surveys and clinical scales.

In addition to opipramol, compositions of the present invention may include prodrugs of opipramol, which are metabolized or converted to yield active opipramol. Prodrugs and other modified forms of opipramol may be employed to increase its solubility and commensurately, its oral bioavailability. Increasing the bioavailability of opipramol may result in reduced inter-subject variability in drug exposure and a more normalized therapeutic delivery and effectiveness.

Prodrugs include compounds of the invention, for example opipramol or opipramol derivatives, wherein one or more appropriate active groups or chemically modifiable moieties of the parent drug have been modified to improve solubility (in physiological solutions, including blood and other tissues and compartments of mammalian subjects), bioavailability, half-life, and/or pharmacological activity in vivo, and generally the subject modification may be reversed upon administration to a mammalian, e.g., human, subject. Reversion is usually achieved by an enzyme naturally present in the subject, such as an endogenous phosphatase, though it is possible for a second agent to be administered together with a prodrug in order to mediate the prodrug reversion to a more active form, most often the parental drug form, in vivo.

Within the methods and compositions of the invention, the solubility, bioavailability, half-life, and/or pharmacological activity in vivo of opipramol can be increased using novel opipramol prodrug derivatives, including but not limited to phosphoester prodrugs of opipramol. In one or more embodiments, the prodrug modifications useful within the invention for opipramol include ester modifications, the parent drug is modified to an ester derivative prodrug form, and reversion may be carried out by an esterase, etc.

Additional prodrug ester derivatives of opipramol and other active compounds for use within the invention are provided herein, which may be produced by additional prodrug ester modifications directed to, for example, the following alternative active groups or moieties of the subject parent compound, alkyl, aryl, arylalkyl, alkoxy, alkoxyl, etc., modified to any of a range of contemplated ester prodrug forms convertible in vivo by reversion to the parent compound, or otherwise converted to yield a modified, active drug compound in vivo. In accordance with this aspect of the invention, additional convertible ester prodrug derivatives of opipramol and other therapeutic compounds for use within the invention will include a range of physiologically hydrolyzable esters, including alkylbenzyl, methoxybenzyl, indanyl, phthalyl, methoxymethyl, alkanoyloxy-alkyl, acetoxymethyl, alkoxycarbonyloxy-alkyl, glycyloxymethyl, phenylglycyloxymethyl, and other physiologically hydrolyzable esters that may be prepared using conventional techniques known in the art.

Useful methods and materials to produce additional prodrug derivatives of opipramol and other therapeutic compounds for use within the invention may be found, for example in Design of Prodrugs, edited by H. Bundgaard, (Elsevier, 1985) and Methods in Enzymology, 112:309-396, edited by K. Widder, et al. (Academic Press, 1985); A Textbook of Drug Design and Development, edited by Krosgaard-Larsen and H. Bundgaard, Chapter 5, “Design and Application of Prodrugs,” by H. Bundgaard, pp. 113-191 (1991); and H. Bundgaard, Advanced Drug Delivery Reviews, 8:1-38 (1992), each incorporated herein by reference.

The solubility, bioavailability and/or pharmacological activity of opipramol can also be increased by modifying the compound to yield salt and other forms of opipramol that have an increased solubility compared to the parent compound. Such modifications include modification with an acid ester. Pharmaceutically acceptable salts of opipramol or other active therapeutic compounds for use within the methods and compositions of the invention include salts formed with inorganic and organic bases. Such salts, including ammonium salts; alkali metal salts, such as lithium, sodium and potassium salts; alkaline earth metal salts, such as calcium and magnesium salts; salts with organic bases, such as amine like salts (e.g., dicyclohexyl amine salt, benzathine, N-methyl-D-glucamine, and hydrabamine salts); and salts with amino acids like arginine, lysine and the like; and zwitterions, the so-called “inner salts”. Pharmaceutically acceptable salts in this context also include acid addition salts, for example salts formed with strong inorganic acids, such as mineral acids, for example sulfuric acid, phosphoric acid or a hydrohalic acid such as HCl or HBr; with strong organic carboxylic acids, such as alkanecarboxylic acids of 1 to 4 carbon atoms which are unsubstituted or substituted, for example, by halogen, for example acetic acid, such as saturated or unsaturated dicarboxylic acids, for example oxalic, malonic, succinic, maleic, fumaric, phthalic or terephthalic acid, such as hydroxycarboxylic acids, for example ascorbic, glycolic, lactic, malic, tartaric or citric acid, such as amino acids, (for example aspartic or glutamic acid or lysine or arginine), or benzoic acid, or with organic sulfonic acids, such as (C.sub.1-4)alkyl or arylsulfonic acids which are unsubstituted or substituted, for example by halogen, for example methanesulfonic acid or p-toluenesulfonic acid.

Prodrug modifications, formation of pharmaceutically acceptable salts, and other modifications of opipramol and other therapeutic compounds described herein yield improved solubility, bioavailability, and/or pharmacological efficacy, e.g., of opipramol, of at least 10%, 20%, 20-30%, up to 30-50%, 50-70%, 100%, 200%, or greater, e.g., as compared to an equivalent dose of the unmodified parent drug, such as opipramol. The improved solubility, bioavailability, and/or pharmacological efficacy of opipramol prodrugs and salts and other modified therapeutic compounds within the invention can be readily determined using standard dissolution assays, pharmacokinetic studies, and/or in vitro and in vivo models of pharmacological activities, including in more detailed aspects known useful physiological models of trauma and stressor-related disorder, such as ASD and PTSD. The increase in bioavailability and/or pharmacological activity of ester and other opipramol prodrugs, opipramol salts, and other modified therapeutic compounds provided herein, as determined e.g., using physiological models, such as animal models and/or human clinical studies, will be at least 10%, 20%, or 20-30%, up to 30-50%, 50-70%, 100%, 200% or greater compared to an equal dose of unmodified opipramol parent drug.

Compositions of the invention may include opipramol or a combination opipramol and one or more psychotherapeutic agents. In one or more embodiments of the invention, opipramol is administered in combination with a drug, which may further alleviate the symptoms of the trauma or stressor related disorder. The drugs may be administered sequentially or concurrently with the opipramol. The drugs include an alpha-1-adrenergic receptor antagonist, a beta-adrenergic antagonist, an anticonvulsant, antipsychotic drug, a benzodiazepine, a glucocorticoid, a glucocorticosteroid, a sertonin 1A receptor active drug, a tricyclic or heterocyclic antidepressant drug, a selective serotonin reuptake inhibitor or a serotonin-norepinephrine reuptake inhibitor.

In a further aspect, the invention is a pharmaceutical composition. The pharmaceutical composition comprises a therapeutically effective amount of opipramol in combination with a drug selected from the group consisting of an alpha-1-adrenergic receptor antagonist, a beta-adrenergic antagonist, an anticonvulsant, antipsychotic drug, a benzodiazepine, a glucocorticoid, a glucocorticosteroid, a sertonin 1A receptor active drug, a tricyclic or heterocyclic antidepressant drug, a selective serotonin reuptake inhibitor or a serotonin-norepinephrine reuptake inhibitor.

In certain embodiments of the invention, an adjunctive psychotherapeutic agent is employed in combination with the opipramol. The psychotherapeutic agent may be selected from known anti-depressant drugs, for example, any species within the broad families of tri-cyclic anti-depressants (TCAs) including, but not limited to, amitriptyline, imipramine, or desipramine; specific monoamine reuptake inhibitors, e.g., selective serotonin reuptake inhibitors (SSRIs) including, but not limited to, fluoxetine, fluvoxamine, sertraline and paroxetine, selective norepinephrine reuptake inhibitors, selective dopamine reuptake inhibitors, multiple monoamine reuptake inhibitors, monoamine oxidase inhibitors (MAOIs), noradrenaline reuptake inhibitors (NRIs), multiple monoamine reuptake inhibitors, e.g., that inhibit both serotonin and norepinephrine reuptake (SNRIs) including, but not limited to, venlafaxine and duloxetine, and indeterminate (atypical) anti-depressants are useful within this aspect of the invention. The psychotherapeutic agent may additionally include atypical antipsychotics including, but not limited to, Aripiprazole, Ziprasidone, Risperidone, Quetiepine, or Olanzapine or anticonvulsants including but not limited to lamotrigine, carbamazepine, oxcarbazepine, valproate, levetriacetam, and topiramate.

Within certain embodiments of the invention, one or more of the anti-depressant drugs is coordinately administered or combinatorially formulated with opipramol to treat a trauma and stressor-related disorder, including but not limited to ASD and PTSD. Single drugs, or multiple drugs from one or more of the indicated drug classes, may be co-administered, simultaneously or sequentially, with the opipramol, which may be combinatorially formulated with the psychotherapeutic therapeutic drug or provided in a separate dosage form.

In other detailed embodiments of combinatorial formulations and coordinate treatment methods of the invention, examples of useful anti-depressant agents include, but are not limited to, one or more of the following: MAOIs, such as phenelzine, nortriptyline, selegiline and tranylcypromine; SSRIs, such as paroxetine, fluoxetine, citalopram, trazodone, fluvozamine and sertraline; Tricyclic anti-depressants, such as amitriptyline, desipramine, clomipramine, doxepine, trimipramine, amoxapine, protripyline and imipramine; Tetracyclic anti-depressants; Norepinephrine uptake inhibitors; Selective noradrenaline reuptake inhibitors; Serotonin and norepinephrine reuptake inhibitors, such as venlafaxine and duloxetine; and other anti-depressant agents such as maprotiline, nefazodone, and bupropion. In additional detailed embodiments the combinatorial formulations and coordinate treatment methods of the invention employ one or more useful psychotherapeutic agents selected from the following: SSRI's, such as Lexapro® (escitalopram HBr; indicated to treat depression and generalized anxiety disorder Celexa® (citalopram), Prozac®, Luvox® (fluvoxamine; also indicated to treat obsessive symptoms), Zoloft® (sertraline; also indicated to treat post-traumatic stress syndrome); Tricyclics, such as Amitriptyline, Desipramine, Nortriptyline; SSNRIs, such as Cymbalta® (Duloxetine), Effexor®, and desvenlafaxine; Tetracyclics, such as Remeron® (mirtazepine); MAOIs, such as Nardil® (phenelzine), and Parnate® (tranylcypromine); Serzone® (nefazodone; a phenylpiperazine); Trazodone® (a triazolopyridine); and Wellbutrin® (bupropion; an aminoketone). In additional detailed embodiments the combinatorial formulations and coordinate treatment methods of the invention employ one or more useful psychotherapeutic agents selected from the following: Amitriptyline; Amoxapine; Aripiprazole; Atomoxetine; Bupropion; Citalopram; Clomipramine; Desipramine; Desvenlafaxine; Dothiepin; Doxepin; Duloxetine; Escitalopram; Fluoxetine; Fluvoxamine; Imipramine; Isocarboxazid; Lofepramine; Maprotiline; Milnacipran; Mirtazapine; Moclobemide; Nefazodone; Notriptyline; Paroxetine; Phenelzine; Protriptyline; Quetiapine; Reboxetine; Selegiline; Sertraline; Tianeptine; Tranylcypromide; Trazodone; Trimipramine; and Venlafaxine.

In other detailed combinatorial formulations and coordinate treatment methods of the present invention, the psychotherapeutic agent is an anxiolytic drug agent including, but not limited to, benzodiazepines, such as alaprazolam, chlordiazepoxide, clonazepam, chlorazepate, diazepam, lorazepam, oxazepam and prazepam; non-benzodiazepine agents, such as buspirone; and tranquilizers, such as barbituates.

Benzodiazepines, anti-depressants, selective serotonin reuptake inhibitors and the azapirone agonist of the serotonin 1A receptor, buspirone (Lydiard et al., 1996) have been used with some success in the treatment of anxiety and anxiety disorders and are therefore contemplated for effective use within the methods and compositions of the invention.

In one or more other detailed combinatorial formulations and coordinate treatment methods of the present invention, the psychotherapeutic agent is one or more steroidal anti-inflammatory agents are chosen from mineralosteroids and glucorticosteroids. In some embodiments, one or more steroidal anti-inflammatory agents are chosen from those having a pregna-1,4-diene-3,20 dione core structure. In one aspect of this embodiment, the one or more steroidal anti-inflammatory agents having a pregna-1,4-diene-3,20 dione core structure are chosen from dexamethasone, fluorometholone, betamethasone, corticosterone, and prednisolone. Examples of steroidal anti-inflammatory agents include, but are not limited to, budesonide, pregnenolone, prednisone, prednisolone, methylprednisolone, triamcinolone, dexamethasone, betamethasone, parametasone, cortisone, and hydrocortisone.

In one or more other detailed combinatorial formulations and coordinate treatment methods of the present invention, the psychotherapeutic agent is one or more glucocorticoid or glucocorticosteroid. The one or more glucocorticoids contained in a composition according to the invention is selected from the group consisting of hydrocortisone, cortisone, prednisolone, prednisone, methylprednisone, triamcinolone, paramethasone, betamethasone, dexamethasone and fludrocortisone including pharmaceutically acceptable esters, salts and complexes thereof.

Examples of the one or more glucocorticoids of the first part (as discussed above) are synthetic glucocorticoids such as, e.g., hydrocortisone 21-succinate, prednisolone, prednisone, methylprednisone, triamcinolone, paramethasone, betamethasone, dexamethasone and fludrocortisone including pharmaceutically acceptable esters, salts and complexes thereof. An especially suitable example is hydrocortisone or hydrocortisone 21-succinate or a pharmaceutically acceptable salt thereof.

As indicated in the sections above, the one or more glucocorticoids may be a mixture of glucocorticoids.

In one or more embodiments, the one or more glucocorticoid is hydrocortisone. In one or more embodiments, the hydrocortisone may be substituted for cortisone. The term “cortisone” includes “cortisone acetate”.

Due to different potencies of the glucocorticoids, the term “hydrocortisone equivalents” is used. The term “hydrocortisone equivalents” is used herein to define the amount in mg of a specific glucocorticoid that corresponds to 1 mg of hydrocortisone for the purpose of systemic glucocorticoid therapy as generally understood by medical practitioners. The term is based on the fact that the individual glucocorticoids have different potencies and in order to achieve a desired therapeutic effect different doses of the individual glucocorticoids are required. For example, in certain embodiments, the equivalent doses of the glucocorticoids can be calculated based on the following table.

TABLE 1

| | | Hydrocortisone equivalent |
|---|---|---|
| | | (1 mg of the glucocorticoid |
| | | corresponds to the |
| | Equivalent amount | listed amount in mg of |
| Glucocorticoid | (mg) | hydrocortisone) |
| Cortisone acetate | 25 | 0.8 |
| Hydrocortisone | 20 | 1 |
| Prednisolone | 5 | 4 |
| Prednisone | 5 | 4 |
| Methylprednisolone | 4 | 5 |
| Triamcinolone | 4 | 5 |
| Pararnethasone | 2 | 10 |
| Betarnethasone | 0.75 | 26.66 |
| Dexamethasone | 0.75 | 26.66 |
| Fludrocortisone | 0.05 | 400 |

The glucocorticoids of the combination pharmaceutical composition should each include a hydrocortisone equivalent daily dose of 5-300 mg. For the purpose of comparison, a table is given herein describing the equivalent milligram dosage of the various glucocorticoids. Thus, other forms of synthetic glucocorticoids in equivalent doses might be used. Normally, a pharmaceutical composition according to the present invention contains a total amount of hydrocortisone equivalents expressed as hydrocortisone in the composition from about 1 to about 300 mg. In specific embodiments, the total amount of hydrocortisone equivalents in the composition is from about 1 to about 300 mg such as, e.g., from about 1 to about 250 mg, from about 5 to about 200 mg, from about 5 to about 150 mg, from about 5 to about 10 mg or from about 10 to about 80 mg.

In some embodiments of the invention, dexamethasone is used in the combination treatments and co-formulations. Dexamethasone is available from a number of sources including Par Pharmaceuticals (Woodcliff Lake, N.J.). In one embodiment, dexamethasone is administered in an amount from about 0.25 mg to about 50 mg per day. In one aspect of this embodiment, dexamethasone is administered in an amount from about 0.25 mg to about 25 mg, 0.25 mg to about 20 mg, 0.25 mg to about 15 mg, 0.25 mg to about 10 mg, or 0.25 mg to about 5 mg per day. In one aspect of this embodiment, dexamethasone is administered in an amount from about 100 or less mg, 90 or less mg, 80 or less mg, 70 or less mg, 60 or less mg, or 50 or less mg per day. In one aspect of this embodiment, dexamethasone is administered in an amount from about 0.5 or more mg, 5 or more mg, 10 or more mg, 20 or more mg, 30 or more mg, 40 or more mg, or 50 or more mg per day.

In some aspects of the invention, budesonide is used in the combination treatments and co-formulations. Budesonide is available from a number of sources including Astrazeneca (Willimgtom, Del.). In one embodiment, budesonide is administered in an amount from about 0.25 mg to about 10 mg per day. In one aspect of this embodiment, budesonide is administered in an amount from about 0.25 mg to about 7.5 mg, 0.25 mg to about 6.5 mg, 0.25 mg to about 5.5 mg, 0.25 mg to about 5.0 mg, or 0.25 mg to about 4.5 mg per day. In one aspect of this embodiment, budesonide is administered in an amount from about 10 or less mg, 9 or less mg, 8 or less mg, 7 or less mg, 6 or less mg, or 5 or less mg per day. In one aspect of this embodiment, cortisone is administered in an amount from about 0.25 or more mg, 1 or more mg, 2 or more mg, 3 or more mg, 4 or more mg, or 5 or more mg per day.

In some aspects of the invention, cortisone is used in the combination treatments and co-formulations. Cortisone is available from a number of sources including Pfizer (NY, N.Y.). In one embodiment, cortisone is administered in an amount from about 10 mg to about 300 mg per day. In one aspect of this embodiment, cortisone is administered in an amount from about 10 mg to about 275 mg, 10 mg to about 250 mg, 10 mg to about 200 mg, 10 mg to about 175 mg, or 10 mg to about 150 mg per day. In one aspect of this embodiment, cortisone is administered in an amount from about 200 or less mg, 150 or less mg, 125 or less mg, 100 or less mg, 90 or less mg, or 80 or less mg per day. In one aspect of this embodiment, cortisone is administered in an amount from about 20 or more mg, 40 or more mg, 60 or more mg, 80 or more mg, 100 or more mg, or 150 or more mg per day.

In some aspects of the invention, betamethasone is used in the combination treatments and co-formulations. Betamethasone is available from a number of sources including Schering-Plough (Kenilworth, N.J.). In one embodiment, betamethasone is administered in an amount from about 0.25 mg to about 25 mg per day. In one aspect of this embodiment, betamethasone is administered in an amount from about 0.25 to about 20 mg, 0.25 mg to about 5.5 mg, 0.25 mg to about 5.0 mg, or 0.25 mg to about 4.5 mg per day. In one aspect of this embodiment, betamethasone is administered in an amount from about 7.5 or less mg, 7 or less mg, 6.5 or less mg, 6 or less mg, 5.5 or less mg, or 5 or less mg per day. In one aspect of this embodiment, betamethasone is administered in an amount from about 0.25 or more mg, 1 or more mg, 2 or more mg, 3 or more mg, 4 or more mg, or 5 or more mg per day.

In some aspects of the invention, hydrocortisone is used in the combination treatments and co-formulations. Hydrocortisone is available from a number of sources including Pfizer (NY, N.Y.). In one embodiment, hydrocortisone is administered in an amount from about 20 mg to about 800 mg per day. In one aspect of this embodiment, hydrocortisone is administered in an amount from about 20 mg to about 700 mg, 20 mg to about 600 mg, 20 mg to about 500 mg, 20 mg to about 400 mg, or 20 mg to about 300 mg per day. In one aspect of this embodiment, hydrocortisone is administered in an amount from about 700 or less mg, 600 or less mg, 500 or less mg, 400 or less mg, 300 or less mg, or 200 or less mg day. In one aspect of this embodiment, hydrocortisone is administered in an amount from about 20 or more mg, 30 or more mg, 40 or more mg, 50 or more mg, 60 or more mg, or 70 or more mg day.

In some aspects of the invention, methylprednisolone is used in the combination treatments and co-formulations. Methylpredisolone is available from a number of sources including Par Pharmaceutical (Woodcliff Lake, N.J.). In one embodiment, methylprednisolone is administered in an amount from about 4 mg to about 160 mg per day. In one aspect of this embodiment, methylprednisolone is administered in an amount from about 4 mg to about 140 mg, 4 mg to about 120 mg, 4 mg to about 100 mg, or 4 mg to about 80 mg per day. In one aspect of this embodiment, methylprednisolone is administered in an amount from about 150 or less mg, 140 or less mg, 130 or less mg, 120 or less mg, 110 or less mg, or 100 or less mg per day. In one aspect of this embodiment, methylprednisolone is administered in an amount from about 4 or more mg, 10 or more mg, 20 or more mg, 30 or more mg, 40 or more mg, or 50 or more mg per day.

In some aspects of the invention, prednisolone is used in the combination treatments and co-formulations. Prednisolone is available from a number of sources including Par Pharmaceuticals (Woodcliff Lake, N.J.). In one embodiment, prednisolone is administered in an amount from about 5 mg to about 200 mg per day. In one aspect of this embodiment, prednisolone is administered 5 mg to about 180 mg, 5 mg to about 150 mg, 5 mg to about 125 mg, or 5 mg to about 100 mg per day. In one aspect of this embodiment, prednisolone is administered in an amount from about 200 or less mg, 150 or less mg, 125 or less mg, 100 or less mg, 90 or less mg, or 80 or less mg per day. In one aspect of this embodiment, prednisolone is administered in an amount from about 5 or more mg, 10 or more mg, 20 or more mg, 30 or more mg, 40 or more mg, or 50 or more mg per day.

In some aspects of the invention, prednisone is used in the combination treatments and co-formulations. Prednisone is available from a number of sources including Watson Pharmaceuticals (Coronona, Calif.). In one embodiment, prednisone is administered in an amount from about 5 mg to about 200 mg per day. In one aspect of this embodiment, prednisone is administered 5 mg to about 180 mg, 4 mg to about 150 mg, 5 mg to about 125 mg, or 4 mg to about 100 mg per day. In one aspect of this embodiment, prednisone is administered in an amount from about 200 or less mg, 150 or less mg, 125 or less mg, 100 or less mg, 90 or less mg, or 80 or less mg per day. In one aspect of this embodiment, prednisone is administered in an amount from about 20 or more mg, 30 or more mg, 40 or more mg, 50 or more mg, 60 or more mg, or 70 or more mg per day.

In some aspects of the invention, triamcinolone is used in the combination treatments and co-formulations. Triamcinolone is available from a number of sources including Astellas Pharma (Deerfield, Ill.). In one embodiment, triamcinolone is administered in an amount from about 2 mg to about 60 mg per day. In one aspect of this embodiment, triamcinolone is administered 2 mg to about 50 mg, 2 mg to about 45 mg, 2 mg to about 35 mg, or 2 mg to about 30 mg per day. In one aspect of this embodiment, triamcinolone is administered in an amount from about 60 or less mg, 50 or less mg, 40 or less mg, 35 or less mg, 30 or less mg, or 25 or less mg per day. In one aspect of this embodiment, triamcinolone is administered in an amount from about 2 or more mg, 5 or more mg, 10 or more mg, 15 or more mg, 20 or more mg, or 25 or more mg day per day.

In some aspects of the invention, pregnenolone is used in the combination treatments and co-formulations. Pregnenolone is available from a number of sources and has a CAS number of [145-13-1]. In one embodiment, pregnenolone is administered in an amount from about 5 mg to about 1000 mg per day. In one aspect of this embodiment, pregnenolone is administered 5 mg to about 500 mg, 5 mg to about 400 mg, 5 mg to about 300 mg, or 5 mg to about 200 mg per day. In one aspect of this embodiment, pregnenolone is administered in an amount from about 200 or less mg, 150 or less mg, 125 or less mg, 100 or less mg, 90 or less mg, or 80 or less mg per day. In one aspect of this embodiment, pregnenolone is administered in an amount from about 20 or more mg, 30 or more mg, 40 or more mg, 50 or more mg, 75 or more mg, or 100 or more mg per day.

In some aspects of the invention DHEA (Dehydroepiandrosterone) is used in the combination treatments and co-formulations. DHEA is available from a number of sources and has a CAS number of [53-43-0]. In one embodiment, pregnenolone is administered in an amount from about 5 mg to about 300 mg per day. In one aspect of this embodiment, DHEA is administered 5 mg to about 250 mg, 5 mg to about 200 mg, 5 mg to about 150 mg, or 5 mg to about 100 mg per day. In one aspect of this embodiment, DHEA is administered in an amount from about 200 or less mg, 150 or less mg, 125 or less mg, 100 or less mg, 90 or less mg, or 80 or less mg per day. In one aspect of this embodiment, prednisone is administered in an amount from about 20 or more mg, 30 or more mg, 40 or more mg, 50 or more mg, 75 or more mg, or 100 or more mg per day.

In some aspects of the invention 7beta-hydroxy epiandrosterone is used in the combination treatments and co-formulations. 7beta-hydroxy epiandrosterone is available from a number of sources. In one embodiment 7beta-hydroxy epiandrosterone is administered in an amount from about 5 mg to about 1000 mg per day. In one aspect of this embodiment, 7beta-hydroxy epiandrosterone is administered 5 mg to about 500 mg, 5 mg to about 400 mg, 5 mg to about 300 mg, or 5 mg to about 200 mg per day. In one aspect of this embodiment, 7beta-hydroxy epiandrosterone is administered in an amount from about 200 or less mg, 150 or less mg, 125 or less mg, 100 or less mg, 90 or less mg, or 80 or less mg per day. In one aspect of this embodiment, 7beta-hydroxy epiandrosterone is administered in an amount from about 20 or more mg, 30 or more mg, 40 or more mg, 50 or more mg, 75 or more mg, or 100 or more mg per day.

The invention further provides additional combination therapy strategies for treating trauma and stressor-related disorders. According to this aspect of the invention, an individual in need of treatment is administered an effective amount of (1) one or more Abeta42 lowering agents, (2) one or more steroidal agents, and (3) one or more compounds selected from the group consisting of NSAIDs, acetylcholine esterase inhibitors (e.g., donepezil, galantamine, rivastagmine), COX-2 inhibitors (cyclooxygenase-2), beta-secretase inhibitors, gamma-secretase inhibitors, NMDA antagonists (i.e., memantine), and GABA-A alpha inverse agonist (see WO 00/27382, WO 96/25948, WO 98/50385 which are herein incorporated by reference in there entireties). NMDA receptor antagonists for combination therapy are memantine, adamantane, amantadine, an adamantane derivative, dextromethorphan, dextrorphan, dizocilpine, ibogaine, ketamine, and remacemide. The invention further encompasses compositions comprising the combination of active ingredients of this aspect of the invention.

The amount, timing and mode of delivery of compositions of the invention comprising an effective amount of a psychotherapeutic compound and an effective amount of opipramol will be routinely adjusted on an individual basis, depending on such factors as weight, age, gender, and condition of the individual, the acuteness of the targeted trauma and stressor-related disorder and/or related symptoms, whether the administration is prophylactic or therapeutic, and on the basis of other factors known to effect drug delivery, absorption, pharmacokinetics, including half-life, and efficacy.

In some embodiments, the compositions and formulations of the present invention may be administered according to a flexible dosing regimen. The treatment regimen provides for dosing periods during which a sufficient number of doses of the compositions and formulations of the present invention are administered to provide relief from trauma and stressor-related disorder symptoms. The one or more embodiments herein provides for dosing according to a discontinuous schedule. In a discontinuous schedule, each dosing period is followed by an evaluation period, during which the user can self-evaluate the occurrence and severity of symptoms. If the user determines that it is necessary to begin a new dosing period, the user may do so at any time following this evaluation period. If, however, the user feels the need to begin a new dosing period before the evaluation period has passed, or to take more than the recommended number of doses, then the user may, for example, choose to seek professional medical advice.

An effective dose or multi-dose treatment regimen for the psychotherapeutic compounds of the invention will ordinarily be selected to approximate a minimal dosing regimen that is necessary and sufficient to substantially prevent or alleviate one or more symptom(s) of the targeted trauma and stressor-related disorder as described herein. For example, opipramol may be administered in dosages ranging from 5 to 500 mg one or more times per day, from 25 to 450 mg per day, from 50 to 400 mg per day, from 50 to 350 mg per day, or from 50 to 300 mg per day. Typically, opipramol will be administered bi-daily. In some embodiments, sub-therapeutic amounts may be used. Exemplary suggested dosage ranges for selected drugs for use within certain embodiments of the invention are provided below, for illustrative purposes. Additional exemplary dosage ranges are provided below for selected drugs formulated for sustained delivery within additional embodiments of the invention, also for illustrative purposes.

These and other effective unit dosage amounts of either or both of the psychotherapeutic agent and/or opipramol may be administered in a single dose, or in the form of multiple daily, weekly or monthly doses, for example in a dosing regimen comprising from 1 to 5, or 2-3, doses administered per day, per week, or per month. In exemplary embodiments, exemplary dosages of selected drugs as illustrated above are administered one, two, three, or four times per day. In more detailed embodiments, specific dosages within the specified exemplary ranges above are administered once, twice, or three times daily. In alternate embodiments, dosages are calculated based on body weight, and may be administered, for example, in amounts as exemplified above adjusted for body weight.

In more detailed embodiments of the invention, the sustained release compositions and dosage forms will yield a therapeutic level of an active therapeutic agent following administration to a mammalian subject in a desired dosage amount (e.g., 25, 50, 100, 150, 200, 250, 300, 350, 400, 450 or 500 mg) that yields a minimum plasma concentration of at least a lower end of a therapeutic dosage range as exemplified herein over a period of at least about 6 hours, at least about 8 hours, at least about 12 hours, at least about 18 hours, or up to 24 hours or longer. In alternate embodiments of the invention, the sustained release compositions and dosage forms will yield a therapeutic level of active therapeutic agent following administration to a mammalian subject in a desired dosage amount (e.g., 25, 50, 100, 150, 200, 250, 300, 350, 400, 450 or 500 mg) that yields a minimum plasma concentration that is known to be associated with clinical efficacy, over a period of at least about 6 hours, at least about 8 hours, at least about 12 hours, at least about 18 hours, or up to 24 hours or longer.

In certain embodiments, the active therapeutic agent is/are released from the compositions and dosage forms of the invention and delivered into the blood plasma or other target site of activity in the subject in a sustained release profile characterized in that from about 0% to 20% of the active compound is released and delivered (as determined, e.g., by measuring blood plasma levels) within in 0 to 2 hours, from 20% to 50% of the active compound is released and delivered within about 2 to 12 hours, from 50% to 85% of the active compound is released and delivered within about 3 to 20 hours, and greater than 75% of the active compound is released and delivered within about 5 to 18 hours.

Pharmaceutical dosage forms of a compound of the present invention may optionally include excipients recognized in the art of pharmaceutical compounding as being suitable for the preparation of dosage units as discussed above. Such excipients include, without intended limitation, binders, fillers, lubricants, emulsifiers, suspending agents, sweeteners, flavorings, preservatives, buffers, wetting agents, disintegrants, effervescent agents and other conventional excipients and additives.

The compositions of the invention for treating trauma and stressor-related disorders, including ASD and PTSD, can thus include any one or combination of the following: a pharmaceutically acceptable carrier or excipient; other medicinal agent(s); pharmaceutical agent(s); adjuvants; buffers; preservatives; diluents; and various other pharmaceutical additives and agents known to those skilled in the art. These additional formulation additives and agents will often be biologically inactive and can be administered to patients without causing deleterious side effects or interactions with the active agent.

By “pharmaceutically acceptable carrier” is meant any diluent or excipient that is compatible with the other ingredients of the formulation, and which is not deleterious to the recipient. The pharmaceutically acceptable carrier can be selected on the basis of the desired route of administration, in accordance with standard pharmaceutical practices. Pharmaceutical compositions of the invention for parenteral administration can take the form of an aqueous or nonaqueous solution, dispersion, suspension or emulsion. In preparing pharmaceutical compositions of the invention for parenteral administration, opipramol can be mixed with a suitable pharmaceutically acceptable carrier such as water, oil (particularly a vegetable oil), ethanol, saline solutions (e.g., normal saline), aqueous dextrose (glucose) and related sugar solutions, glycerol, or glycols such as propylene glycol or polyethylene glycol. Pharmaceutical compositions of the invention for parenteral administration preferably contain a water-soluble salt of opipramol. Stabilizing agents, antioxidizing agents and preservatives can also be added to the pharmaceutical compositions for parenteral administration. Suitable antioxidizing agents include sulfite, ascorbic acid, citric acid and its salts, and sodium EDTA. Suitable preservatives include benzalkonium chloride, methyl- or propyl-paraben, and chlorbutanol.

In preparing pharmaceutical compositions of the invention for oral administration, opipramol can be combined with one or more solid or liquid inactive ingredients to form tablets, capsules, pills, powders, granules or other suitable oral dosage forms. For example, opipramol can be combined with at least one pharmaceutically acceptable carrier such as a solvent, filler, binder, humectant, disintegrating agent, solution retarder, absorption accelerator, wetting agent absorbent or lubricating agent. In one embodiment, opipramol is combined with carboxymethylcellulose calcium, magnesium stearate, mannitol or starch, and is formed into tablets by conventional tableting methods.

Pharmaceutical compositions of the invention can be formulated so as to provide buccal absorption including thin film formulations and orally dissolving tablets to provide faster absorption than the oral/GI route and to bypass first-pass hepatic metabolism of opipramol by cytochrome P-450 3A4 as a CYP3A substrate. Preferably, a controlled-release pharmaceutical composition of the invention is capable of releasing opipramol into a subject at a rapid onset, so as to maintain a substantially constant or desired pharmacological activity for a given period of time, reduce or remove the effect of food on absorption, and to provide elimination of the drug and metabolites from the body with a reduced terminal elimination phase.

Pharmaceutical compositions of the invention can also be formulated so as to provide controlled-release of opipramol upon administration of the composition to a subject. Preferably, a controlled-release pharmaceutical composition of the invention is capable of releasing opipramol into a subject at a desired rate, so as to maintain a substantially constant or desired pharmacological activity for a given period of time. As used herein, a “controlled-release component” is a compound such as a lipid or mixture of lipids, liposome and/or microsphere that induces the controlled-release of opipramol into the subject upon exposure to a certain physiological compound or condition. For example, the controlled-release component can be biodegradable, activated by exposure to a certain pH or temperature, by exposure to an aqueous environment, or by exposure to enzymes.

Formulation of controlled-release pharmaceutical compositions of the invention is within the skill in the art. Controlled release formulations suitable for use in the present invention are described in, for example, U.S. Pat. No. 5,674,533 (liquid dosage forms), U.S. Pat. No. 5,591,767 (liquid reservoir transdermal patch), U.S. Pat. No. 5,120,548 (device comprising swellable polymers), U.S. Pat. No. 5,073,543 (ganglioside-liposome vehicle), U.S. Pat. No. 5,639,476 (stable solid formulation coated with a hydrophobic acrylic polymer), the entire disclosures of which are herein incorporated by reference.

Biodegradable microparticles can also be used to formulate controlled-release pharmaceutical compositions suitable for use in the present invention, for example as described in U.S. Pat. Nos. 5,354,566 and 5,733,566, the entire disclosures of which are herein incorporated by reference.

In one embodiment, controlled-release pharmaceutical compositions of the invention comprise opipramol and a controlled-release component. As used herein, a “controlled-release component” is a compound such as a polymer, polymer matrix, gel, permeable membrane, liposome and/or microsphere that induces the controlled-release of opipramol into the subject upon exposure to a certain physiological compound or condition. For example, the controlled-release component can be biodegradable, activated by exposure to a certain pH or temperature, by exposure to an aqueous environment, or by exposure to enzymes. An example of a controlled-release component which is activated by exposure to a certain temperature is a sol-gel. In this embodiment, opipramol is incorporated into a sol-gel matrix that is a solid at room temperature. This sol-gel matrix is implanted into a subject having a body temperature high enough to induce gel formation of the sol-gel matrix, thereby releasing the active ingredient into the subject.

In one embodiment, pharmaceutical compositions of the invention may comprise opipramol and components that form micelles. Micelles containing opipramol in the stomach and proximal small intestine facilitate absorption. Example of a micelle-component which is activated by exposure to a certain temperature is found in U.S. Pat. Nos. 6,761,903; 6,720,001; 6,383,471; 6,309,663; 6,267,985; and 6,248,363, incorporated herein by reference. In this embodiment, opipramol is incorporated into a soft-gel capsule. Such components may mimic the augmentation of absorption termed the “food effect”, and such formulations may provide more predictable absorption by eliminating the “food effect” from dietary sources.

The composition of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The magnitude of a prophylactic or therapeutic dose of the active ingredient (i.e., opipramol or metabolite thereof) in the prevention or treatment of a human will vary with the type of affliction, the severity of the patient's affliction and the route of administration. The dose and dose frequency will also vary according to the age, weight and response of the individual patient. However, the dosage will not equal or exceed 500 mgs per day. In a preferred embodiment, one dose is given at bed time or up to several hours before bedtime to facilitate the achievement of deep, refreshing sleep. Bedtime may be any hour of the day at which a person engages in the most extensive period of sleep.

Any of the methods of treatment described above may be combined with psychotherapeutic intervention to improve the outcome of the treatment. Of particular interest is psychotherapeutic intervention directed at either modifying traumatic memories reducing emotional responses to traumatic memories, and including: psychological debriefing, cognitive behavior therapy and eye movement desensitization and reprocessing, systematic desensitization, relaxation training, biofeedback, cognitive processing therapy, stress inoculation training, assertiveness training, exposure therapy, combined stress inoculation training and exposure therapy, combined exposure therapy and relaxation training and cognitive therapy. In each case, the goal of the intervention involves either modifying traumatic memories or reducing emotional responses to traumatic memories. The intended result is generally improvement as evidenced in terms of reducing intrusive combat memories, physiological responding, anxiety, depression and feelings of alienation.

An active therapeutic agent of the present invention will often be formulated and administered in an oral dosage form, optionally in combination with a carrier or other additive(s). Suitable carriers common to pharmaceutical formulation technology include, but are not limited to, microcrystalline cellulose, lactose, sucrose, fructose, glucose dextrose, or other sugars, di-basic calcium phosphate, calcium sulfate, cellulose, methylcellulose, cellulose derivatives, kaolin, mannitol, lactitol, maltitol, xylitol, sorbitol, or other sugar alcohols, dry starch, dextrin, maltodextrin or other polysaccharides, inositol, or mixtures thereof. Exemplary unit oral dosage forms for use in this invention include tablets and capsules, which may be prepared by any conventional method of preparing pharmaceutical oral unit dosage forms can be utilized in preparing oral unit dosage forms. Oral unit dosage forms, such as tablets or capsules, may contain one or more conventional additional formulation ingredients, including, but are not limited to, release modifying agents, glidants, compression aides, disintegrants, lubricants, binders, flavors, flavor enhancers, sweeteners and/or preservatives. Suitable lubricants include stearic acid, magnesium stearate, talc, calcium stearate, hydrogenated vegetable oils, sodium benzoate, leucine carbowax, magnesium lauryl sulfate, colloidal silicon dioxide and glyceryl monostearate. Suitable glidants include colloidal silica, fumed silicon dioxide, silica, talc, fumed silica, gypsum and glyceryl monostearate. Substances which may be used for coating include hydroxypropyl cellulose, titanium oxide, talc, sweeteners and colorants. The aforementioned effervescent agents and disintegrants are useful in the formulation of rapidly disintegrating tablets known to those skilled in the art. These typically disintegrate in the mouth in less than one minute, and preferably in less than thirty seconds. By effervescent agent is meant a couple, typically an organic acid and a carbonate or bicarbonate. Such rapidly acting dosage forms would be useful, for example, in the prevention or treatment of acute attacks of panic disorder.

The active therapeutic agent of the invention can be prepared and administered in any of a variety of delivery forms known in the art. Compositions and methods of the invention are provided for topical administration of an active therapeutic agent of the present invention for treating trauma and stressor-related disorder including ASD and PTSD. Topical compositions may comprise a compound of the present invention and any other active or inactive component(s) incorporated in a dermatological or mucosal acceptable carrier, including in the form of aerosol sprays, powders, dermal patches, sticks, granules, creams, pastes, gels, lotions, syrups, ointments, impregnated sponges, cotton applicators, or as a solution or suspension in an aqueous liquid, non-aqueous liquid, oil-in-water emulsion, or water-in-oil liquid emulsion. These topical compositions may comprise a compound of the present invention dissolved or dispersed in water or other solvent or liquid to be incorporated in the topical composition or delivery device. It can be readily appreciated that the transdermal route of administration may be enhanced by the use of various dermal penetration enhancers known to those skilled in the art. Formulations suitable for such dosage forms incorporate excipients commonly utilized therein, particularly means, e.g. structure or matrix, for sustaining the absorption of the drug over an extended period of time, for example 24 hours. A once-daily transdermal patch will be particularly useful for patients suffering from or at risk for selected trauma and stressor-related disorders, such as generalized anxiety disorder, acute stress disorder or PTSD.

Use of transdermal delivery devices and methods is particularly advantageous for administration of opipramol within the present invention. Transdermal administration of opipramol may provide several advantages over oral delivery, including improved patient compliance and responsivity, bypass of first-pass metabolism, sustained drug delivery, and minimal variability both within and between patients. The properties of opipramol indicate that it is suitable for formulation in a transdermal patch.

A matrix-type transdermal patch developed for opipramol treatment of trauma and stressor-related disorders may be effective for opipramol treatment of acute stress disorder and PTSD. The dose of opipramol delivered by transdermal patch will typically range from 5-500 mg per day, from 25 to 450 mg per day, from 40 to 450 mg per day, from 50 to 400 mg per day, from 50 to 300 mg per day, from 100 to 300 mg per day, from 150 to 300 mg per day, as determined by the prescribing physician in light of such ordinary dosing factors as patient condition, body weight, duration of treatment, etc. Transdermal patch delivery devices and methods of the invention will yield increased opipramol bioavailability compared to oral administration of an equivalent opipramol dose. In particular, a transdermal patch will result in a 20-50% bioavailability increase, more typically a 50%-100% increase, up to a two-fold, three-fold, or greater increase, and as much as a five-fold or greater increase in bioavailability compared to that obtained with an equivalent oral dosage form (e.g., an IR or SR capsule or tablet form). The pharmacokinetic properties of oral and transdermal patch delivered opipramol can be measured as described in Example 6. Within more detailed aspects of the invention, transdermal delivery devices and methods for delivering opipramol and other therapeutic compounds as described herein will yield approximately zero-order kinetics, to provide steady state levels of the active drug within 24 hours, and to reduce peak to trough ratios of drug levels (e.g., reduced Cmax/Cmin) in comparison to oral delivery, to yield more continuous therapeutic exposure.

Transdermal delivery systems useful for the compositions and methods of the present invention are typically fabricated as multilayered polymeric laminates in which a drug reservoir or a drug-polymer matrix is sandwiched between two polymeric layers: an outer impervious backing layer that creates an occlusive environment and prevents the loss of drug through the backing surface and an inner polymeric layer that functions as an adhesive and/or rate-controlling membrane. In the case of a drug reservoir design, the reservoir is sandwiched between the backing and a rate controlling membrane. The drug releases only through the rate-controlling membrane, which can be microporous or nonporous. In the drug reservoir compartment, the drug can be in the form of a solution, suspension, or gel or dispersed in a solid polymer matrix. On the outer surface of the polymeric membrane a thin layer of drug-compatible, hypoallergenic adhesive polymer may be applied.

For the drug matrix design, two general types of system include the drug-in-adhesive system and the matrix dispersion system. In the drug-in-adhesive system, the drug reservoir is formed by dispersing the drug in an adhesive polymer and then spreading the medicated polymer adhesive by solvent casting or by melting the adhesive (in the case of hot-melt adhesives) onto an impervious backing layer. On top of the reservoir, layers of unmedicated adhesive polymer are applied. In the case of the matrix dispersion system, the drug is dispersed homogeneously in a hydrophilic or lipophilic polymer matrix and fixed onto a drug-impermeable backing layer by solvent casting or extrusion. Instead of applying the adhesive on the face of the drug reservoir, it is applied to form a peripheral adhesive. Illustrative examples of suitable adhesives as matrix type delivery systems include those described in U.S. Pat. Nos. 5,474,783, and 5,656,386. Other transdermal systems include films, plasters, dressings, and bandages, as well as multilayer delivery systems in which the drug is solubilized or contained in one or more separate layers, and reservoir-type delivery systems in which the drug is solubilized or contained in a reservoir or depot separate from the adhesive which attaches directly to the skin or mucosa.

Suitable adhesives for transdermal patches are known in the art and include pressure-sensitive adhesives and bioadhesives. Pressure sensitive adhesives suitable for use in accordance with the invention include, but are not limited to, pressure-sensitive silicone adhesives, pressure-sensitive acrylic adhesives, and mixtures of any two or more thereof. Exemplary pressure-sensitive silicone adhesives include polysiloxanes and other silicone adhesives as disclosed in U.S. Pat. Nos. 4,591,622; 4,584,355; 4,585,836; 4,655,767; and 5,958,446. Suitable silicone pressure-sensitive adhesives are commercially available and include the silicone adhesives sold under the trademarks BIO-PSA X7-3027. BIO-PSA X7-4919, BIO-PSA X7-2685, and BIO-PSA X7-3122 by Dow Corning Corporation, Medical Products, Midland, Mich.

Bioadhesive materials useful in some embodiments include those described in U.S. Pat. No. 6,562,363. For example, bioadhesive materials may include polymers, either water soluble or water insoluble, with or without crosslinking agents, which are bioadhesive. Exemplary bioadhesives include natural materials, cellulose materials, synthetic and semi-synthetic polymers, and generally, any physiologically acceptable polymer showing bioadhesive properties, or mixtures of any two or more thereof.

Suitable acrylic-based pressure-sensitive adhesives for transdermal patches are also known in the art. Such acrylic-based polymers may be used as the primary pressure-sensitive adhesive (see, e.g., U.S. Pat. No. 4,390,520), or may be used in combination with other polymers which may or may not be pressure-sensitive adhesives (see, e.g. U.S. Pat. No. 4,994,267). Acrylic-based pressure-sensitive adhesives may be polymerized with functional monomers to provide functional groups on the acrylic-based adhesive, such as may be desired to improve wear properties and drug delivery. Suitable polyacrylic acid polymers include polymers of acrylic acid crosslinked with polyalkenenyl ethers (generically known as carbomers) or divinyl glycol (generically known as polycarbophils).

Polymer blends as described in U.S. Pat. No. 5,958,446 may also be used as pharmaceutically acceptable carriers and adhesives in the transdermal compositions embodied herein.

In certain embodiments of transdermal patches used in the compositions of the invention, a plasticizer or tackifying agent is incorporated into the formulation to improve the adhesive characteristics of the composition. A tackifying agent is particularly useful in those embodiments in which the drug does not plasticize the polymer. Suitable tackifying agents are those known in the art including: (1) aliphatic hydrocarbons; (2) mixed aliphatic and aromatic hydrocarbons; (3) aromatic hydrocarbons; (4) substituted aromatic hydrocarbons; (5) hydrogenated esters; (6) polyterpenes; and (7) hydrogenated wood rosins. The tackifying agent employed is preferably compatible with the blend of polymers. In some embodiments, the tackifying agent is silicone fluid (e.g., 360 Medical Fluid, available from Dow Corning Corporation, Midland, Mich.) or mineral oil. Silicone fluid is useful for blends comprising polysiloxane as a major component. In other embodiments, where polyacrylate, for example, is a major component, mineral oil may be used as a tackifying agent.

Transdermal patch compositions may also contain agents known to accelerate the delivery of the drug through the skin. Such agents have been referred to as skin-penetration enhancers, accelerants, adjuvants, and sorption promoters. This class of agents includes those with diverse mechanisms of action including those which have the function of improving the solubility and diffusibility of the drug within the multiple polymer and those which improve percutaneous absorption, for example, by changing the ability of the stratum corneum to retain moisture, softening the skin, improving the skin's permeability, acting as penetration assistants or hair-follicle openers or changing the state of the skin including the boundary layer. Some of these agents have more than one mechanism of action, but in essence they serve to enhance the delivery of the drug. Some exemplary agents are listed in U.S. Pat. Nos. 5,958,446 and 6,562,363.

The topical or transdermal compositions of the present invention can be made in accordance with methods known in the art. For example, opipramol can be blended with the pharmaceutically acceptable topical or transdermal carrier components, or by dissolving the opipramol in a solvent and combining the solution with the pharmaceutically acceptable topical or transdermal carrier components, or by other conventional methods. In the case of a transdermal patch, the substrate is laminated to one or more additional layers, such as a protective layer, a backing layer, a rate-controlling layer, a membrane layer, or one or more other types of layers known in the art.

The pharmaceutical compositions and dosage forms of the current invention will typically be provided for administration in a sterile or readily sterilizable, biologically inert, and easily administered form.

In other embodiments the invention provides pharmaceutical kits for reducing, treating, preventing or alleviating symptoms in a human subject suffering from or at risk for a trauma and stressor-related disorder. The kits comprise an active therapeutic agent in an effective amount, and a container means for containing the active therapeutic agent for coordinate administration to the subject (for example a container, divided bottle, or divided foil pack). The container means can include a package bearing a label or insert that provides instructions for multiple uses of the kit contents to treat the trauma and stressor-related disorder and reduce symptoms in the subject. In more detailed embodiments, the active therapeutic agent are admixed or co-formulated in a single, combined dosage form, for example a liquid or solid oral dosage form. In alternate embodiments, the active therapeutic agents are contained in the kit in separate dosage forms for coordinate administration. Other variations of memory aids will be readily apparent.

All publications, patents and patent applications cited herein, whether supra or infra, are hereby incorporated by reference in their entirety to the same extent as if each individual publication, patent or patent application was specifically and individually indicated as incorporated by reference. It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein, will only be incorporated to the extent that no conflict arises between that incorporated material and the existing disclosure material.

It must be noted that, as used in this specification and the appended claims, the singular forms “a,” “an” and “the” include plural referents unless the content clearly dictates otherwise. Thus, for example, reference to a “colorant agent” includes two or more such agents.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

As will be appreciated by one having ordinary skill in the art, the methods and compositions of the invention substantially reduce or eliminate the disadvantages and drawbacks associated with prior art methods and compositions.

It should be noted that, when employed in the present disclosure, the terms “comprises,” “comprising,” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

While it is apparent that the illustrative embodiments of the invention herein disclosed fulfill the objectives stated above, it will be appreciated that numerous modifications and other embodiments may be devised by one of ordinary skill in the art. Accordingly, it will be understood that the appended claims are intended to cover all such modifications and embodiments, which come within the spirit and scope of the present invention.

What is claimed is:

1. A method for preventing, ameliorating or alleviating symptoms associated with trauma and stressor-related disorders, in which exposure to a traumatic or stressful event is listed explicitly as a diagnostic criterion in a human subject suffering from or at risk for disorders including reactive attachment disorder, disinhibited social engagement disorder, post-traumatic stress disorder (PTSD), acute stress disorder, and adjustment disorders comprising administering to a human in need of such treatment a composition comprising opipramol in an amount effective to prevent, ameliorate or alleviate one or more symptoms of the trauma and stressor-related disorder.

2. The method of claim 1, wherein the opipramol are administered prior to a traumatic event.

3. The method of claim 1, wherein the opipramol are administered after a traumatic event.

4. The method of claim 1, wherein the opipramol are administered after development of symptoms of the trauma and stressor-related disorder.

5. The method of claim 1, wherein the opipramol is administered by a transdermal patch.

6. The method of claim 5, wherein the opipramol is administered in a symptom reducing effective dosage of from about 50 to about 300 mg per day.

7. The method of claim 6, wherein the method further comprises coordinately administering a psychotherapeutic agent in an amount effective to prevent, ameliorate or alleviate one or more symptoms of the disorder.

8. The method of claim 7, wherein the one or more symptoms of the disorder is selected from the group consisting of dissociation, panic, persistent worry, doubt, dread, fear, uneasiness, obsessive thoughts, repeated thoughts, flashbacks of traumatic experiences, mood instability, agitation, restlessness, dyspepsia, headaches, dyspnea, nightmares, ritualistic behaviors, insomnia, cold or sweaty hands and/or feet, shortness of breath, palpitations, hyper alertness, exaggerated startle response, avoidance of particular activities, avoidance of particular thoughts, diminished intensity of feelings, dry mouth, numbness or tingling in the hands or feet, nausea, muscle tension, or dizziness.

9. The method of claim 7, wherein the psychotherapeutic agent is an anti-depressant drug.

10. The method of claim 9, wherein the anti-depressant drug is tri-cyclic anti-depressants (TCAs), specific monoamine reuptake inhibitors, selective serotonin reuptake inhibitors (SSRIs), selective norepinephrine reuptake inhibitors, selective dopamine reuptake inhibitors, multiple monoamine reuptake inhibitors, monoamine oxidase inhibitors (MAOIs), or indeterminate (atypical) anti-depressants.

11. The method of claim 7, wherein the psychotherapeutic agent is administered in a dosage of from about 60 mg to about 1000 mg.

12. The method of claim 11, wherein the psychotherapeutic agent is hydrocortisone.

13. The method of claim 12, wherein the hydrocortisone is administered in a dosage of from about 25 mg to about 300 mg.

14. The method of claim 7, wherein the psychotherapeutic agent and the opipramol are administered to the subject simultaneously.

15. The method of claim 14, wherein psychotherapeutic agent and the opipramol are administered in a single, combined transdermal dosage form.

16. A pharmaceutical composition for preventing, ameliorating or alleviating symptoms associated with trauma and stressor-related disorders, in which exposure to a traumatic or stressful event is listed explicitly as a diagnostic criterion in a human subject suffering from or at risk for disorders including reactive attachment disorder, disinhibited social engagement disorder, post-traumatic stress disorder (PTSD), acute stress disorder, and adjustment disorders comprising opipramol in an amount effective to treat the disorder, wherein the psychotherapeutic agent and the opipramol are admixed or co-formulated in a single, combined dosage form.

17. The pharmaceutical composition according to claim 16, wherein the opipramol is in a symptom reducing effective dosage of about 50 to about 300 mg per day.

18. The pharmaceutical composition according to claim 17, wherein the opipramol is formulated for administration by a transdermal patch mode of delivery.

19. The pharmaceutical composition according to claim 18, wherein the composition further comprises a psychotherapeutic agent in an amount effective to reduce symptoms in the subject.

20. The pharmaceutical composition according to claim 19, wherein the psychotherapeutic therapeutic agent is selected from the group consisting of anti-depressant, anxiolytic, anticonvulsant, antipsychotic, antiaddictive, appetite suppressant drugs and opiate agonists.

21. The pharmaceutical composition according to claim 20, wherein the psychotherapeutic agent comprises from about 60 mg to about 1000 mg.

22. The pharmaceutical composition according to claim 20, wherein the psychotherapeutic therapeutic agent is an anti-depressant drug is selected from the group consisting of tri-cyclic anti-depressants (TCAs), specific monoamine reuptake inhibitors, selective serotonin reuptake inhibitors (SSRIs), selective norepinephrine reuptake inhibitors, selective dopamine reuptake inhibitors, multiple monoamine reuptake inhibitors, monoamine oxidase inhibitors (MAOIs), and indeterminate (atypical) anti-depressants.

23. The pharmaceutical composition according to claim 21, wherein the psychotherapeutic agent is hydrocortisone.

24. The pharmaceutical composition according to claim 22, wherein the hydrocortisone is administered in a dosage of from about 25 mg to about 300 mg.
